(19) United States
(12) Patent Application Publication
(10) Pub. No.: US 2025/0215228 A1
(43) Pub. Date: Jul. 3, 2025

(54) THERMO-REVERSIBLE CONDUCTING HYDROGELS AND THEIR USE FOR EPIDERMAL ELECTRODES OR STANDALONE TRANSMITTER

(71) Applicant: KHALIFA UNIVERSITY OF SCIENCE AND TECHNOLOGY, Abu Dhabi (AE)

(72) Inventors: NAZMI BASIM NAZMI ALSAAFEEN, Abu Dhabi (AE); ANNA MARIA PAPPA, Abu Dhabi (AE); CHARALAMPOS PITSALIDIS, Abu Dhabi (AE); AHSAN HABIB KHANDOKER, Abu Dhabi (AE)

(73) Assignee: KHALIFA UNIVERSITY OF SCIENCE AND TECHNOLOGY, Abu Dhabi (AE)

(21) Appl. No.: 18/621,537

(22) Filed: Mar. 29, 2024

(30) Foreign Application Priority Data
Dec. 29, 2023 (GR) 20230101087

(51) Int. Cl. C08L 89/00; A61B 5/259; A61B 5/266; A61N 1/04; C08L 5/08

(52) U.S. Cl. CPC C08L 89/00; A61B 5/259; A61B 5/266; C08L 5/08; A61N 1/0496; C08L 2201/06; C08L 2203/02; C08L 2203/20; C08L 2205/03

(57) ABSTRACT

It is disclosed new conducting and stretchable hydrogels and “one-pot” process to making them from natural and eco-friendly components, including gelatin, chitosan, and glycerol. Various conducting materials, such as poly(3,4-ethylenedioxythiophene) polystyrene sulfonate (PEDOT:PSS), thermally reduced graphene (TRG), or MXene are introduced in the hydrogels to enhance their conductivity. The resulting conducting hydrogels exhibit remarkable robustness, do not require crosslinking agent, and possess a unique thermo-reversible property, simplifying the fabrication process and ensuring enhanced long-term stability. Moreover, their fabrication is sustainable, employing environmental-friendly materials and processes, while retaining their skin-friendly characteristics. It is also disclosed hydrogel electrodes that were tested for ECG signal acquisition and outperformed the commercial electrodes. The hydrogel-based electrodes deliver high-quality ECG signals, boasting a superior signal-to-noise ratio (SNR) and remarkable resilience against interference and motion artifacts, compared to their commercial AgCl counterparts.

### CROSS-REFERENCE TO RELATED APPLICATION(S)

This application claims priority to Greek patent application no. 20230101087, filed Dec. 29, 2023, which is incorporated herein by reference in its entirety.

### FIELD OF THE INVENTION

The present disclosure relates to hydrogels suitable for use as conductive compositions, methods of making these compositions, and the use of these compositions with medical electrodes, standalone transmitters or the like.

### BACKGROUND OF THE INVENTION

Epidermal electronics have emerged as non-invasive, compact, and ergonomic tools to monitor an individual's health status in resting and during dynamic conditions such as sweating or exercising [1]. The commercial electrophysiology devices are typically metal electrodes such as silver/silver chloride (Ag/AgCl), which although highly conductive, are flat and rigid hence not adequately conforming to human skin. This mismatch often leads to skin irritation, electrode degradation and sweat-induced failure, all of which hampers signal quality in the long term [2, 3]. Furthermore, those electrodes have a finite shelf-life [4] due to the gradual conversion of Ag to AgCl over time, even during storage, severely impacting the stability and reliability of the electrodes.

Conducting hydrogels combine the soft and stretchable nature, water-absorption and retention properties of hydrogels with the conductivity typically provided by conducting polymers or carbon-based materials, making them highly suitable for advanced and comfortable wearable devices [5]. These hydrogels possess inherent adhesiveness, superior skin conformability, and exhibit mixed conduction (both ionic and electronic), along with remarkable mechanical properties, surpassing the performance of conventional metal electrodes. As such, they serve as a more biomimetic alternative for sensing, stimulating, and modulating electrical activity in biological tissues [6]. Furthermore, conducting hydrogels conform to the skin reducing the gap between the electrode and the skin, minimizing skin-electrode contact impedance hence enhancing the signal-to-noise ratio (SNR) of electrophysiological measurements [7, 8].

However, several aspects need to be addressed for clinical and commercial translation to fully realize the potential of conducting hydrogels. The main challenge is optimizing the electrical conductivity to be comparable with metal electrodes while ensuring long-term stability and biocompatibility. To render hydrogels electrically conducting, standard methods include doping them with ionic liquids, metallic nanoparticles, carbon-based conductive fillers or conducting polymers [9-11]. Furthermore, developing scalable fabrication methods while keeping the cost of materials and processes as low as possible is another critical requirement [12].

Hydrogels can be synthesized using various methods, such as polymerization [13], crosslinking [11, 14], solution mixing [11, 15], radiation [16, 22], etc. They can be made from synthetic materials, including polyvinyl alcohol, polyacrylamide and polyethylene glycol [1, 5, 13], natural materials including gelatin, chitosan and alginate [17-21], or hybrids [17, 22, 23].

While synthetic hydrogels might seem superior in terms of mechanical properties they often suffer in terms of biocompatibility, biodegradability, and sustainability. Natural hydrogels are arguably more desirable especially given their high resemblance to the skin, low toxicity, sustainability and facile processing [17, 24]. However, they still need the addition of chemical crosslinkers, often toxic, as they tend to degrade in water and suffer in terms of mechanical robustness and stability.

There is thus a need for new eco-friendly, natural and biocompatible hydrogels, and a method for making the same, that can be used, for instance, for the manufacturing of medical electrodes (e.g., ECG, EMG, EEG) or as a standalone transmitter for various signals.

### SUMMARY OF THE INVENTION

The shortcomings of the prior art are generally mitigated by a new system and method for pre-heating an electrolytic cell typically used for the electrolytic production of aluminum.

According to a first aspect, it is disclosed a thermoreversible electrically and/or ionically conducting hydrogel comprising: a mixture of glycerol, chitosan and gelatin; a given amount of an electrically conducting material, and an aqueous solvent; wherein the conducting hydrogel is free of crosslinking agent.

According to a preferred embodiment, for 12 mL of said aqueous solvent, such as water, the hydrogel comprises:
about 12.5 to 30 v/v % of glycerol;
about 12.5 to 50 v/v % of a chitosan solution, wherein said chitosan solution comprises about 0.5 to 5 wt/Vol. % of chitosan in water with preferably 1 to 3 v/v of 99% acetic acid; and
about 14.5 to 58 wt.vol % of gelatin;
and about 10 v/v % of said conducting material.

According to a preferred embodiment, for 12 mL of said aqueous solvent, such as water, the hydrogel comprises:
about 1.5 to 3 mL of glycerol;
about 1.5 to 3 mL of a chitosan solution, wherein said chitosan solution comprises about 0.5 to 5 wt/Vol. % of chitosan in water with preferably 1 to 3 v/v of 99% acetic acid; and
about 1.75 to 3.75g of gelatin;
and about 10 v/v % of said conducting material.

More preferably, wherein for 12 mL of said aqueous solvent, such as water, the hydrogel comprises:
about 3 mL of glycerol;
about 3 mL of a chitosan solution, wherein said chitosan solution comprises about 0.5 to 5 wt/Vol. % of chitosan in water with preferably 1 to 3 v/v of 99% acetic acid; and
about 3.75g of gelatin;
and about 10 v/v % of said conducting material.

According to a preferred embodiment, said conducting material comprises poly(3,4-ethylenedioxythiophene) polystyrene sulfonate (PEDOT:PSS), thermally reduced graphene (TRG), or Ti3C2Tx (MXene).

According to a preferred embodiment, the hydrogel may be for use for the manufacturing of a medical electrode, a standalone transmitter for various signals, and/or wearable electronics for recording/stimulation. Preferably, the medical electrode or standalone transmitter is manufactured using 3D printing, casting or any sorts of molding techniques. More preferably, the medical electrode is used for electrophysiological signal comprising, but not limited to, electrocardiogramalectromyography (EMG), electroencephalography (EEG), electrogastrography (EGG) or electrooculography (EOG).

According to another aspect, it is disclosed a process for the making of the electrically and/or ionically conducting hydrogel as disclosed herein, the process being a “one-pot” process and comprising the steps of:
heating an aqueous solvent, such as water, at a temperature between about 25 and 60° C.;
adding glycerol and chitosan into the heated solvent and stirring until to form a first mixture;
adding gelatin to the first mixture and stirring until to form a second mixture;
adding a given amount of an electrically conducting material, preferably in the form of salts or ionic liquid, to the second mixture and stirring until to form a third mixture; and
cooling the second or third mixture until the hydrogel is formed.

According to a preferred embodiment, the temperature of the aqueous solvent is about 50-60°, more preferably about 56° C. (i.e. hot plate setting).

According to a preferred embodiment, the aqueous solvent has a pH between about 4 and 6.5.

According to a third aspect, it is disclosed a medical electrode comprising:
the electrically and/or ionically conducting hydrogel as disclosed herein, or obtained by the process as disclosed herein, the conducting hydrogel being in its gel state and having a shape configured for being applied to a patient's skin; and
an electrical connector, preferably comprising a metal or an intermediate flexible substrate, operatively connected to the electrically and/or ionically conducting hydrogel.

According to a preferred embodiment, the shape of the hydrogel is obtained by molding said hydrogel in its sol state at a given temperature, the hydrogel being then cooled until jellification/stretchable (Gel state).

Preferably, the shape of the hydrogel may be obtained by 3D printing said hydrogel in its sol state at a given temperature.

According to a preferred embodiment, the medical electrode as disclosed herein is for use for electrophysiological signal comprising, but not limited to, electrocardiography (ECG), electromyography (EMG), electroencephalography (EEG), electrogastrography (EGG) or electrooculography (EOG).

The resulting conducting hydrogels exhibit remarkable robustness, do not require crosslinking agents, and possess a unique thermo-reversible property, simplifying the fabrication process and ensuring enhanced long-term stability. Moreover, their fabrication is sustainable, employing environmental-friendly materials and processes, while retaining their skin-friendly characteristics.

It is also disclosed hydrogel electrodes that were tested for ECG signal acquisition and outperformed the commercial electrodes. The hydrogel-based electrodes deliver high-quality ECG signals, boasting a superior signal-to-noise ratio (SNR) and remarkable resilience against interference and motion artifacts, compared to their commercial counterparts.

Other advantages of the invention will be disclosed herein after.

### BRIEF DESCRIPTION OF THE DRAWINGS

The above and other aspects, features and advantages of the invention will become more readily apparent from the following description, reference being made to the accompanying drawings in which:

FIG. 1(a) is a schematic showing one-pot synthesis method of the conducting hydrogels as well as an illustration of the hydrogel facile mouldability and appearance at both states (Gel and Sol), according to a preferred embodiment;

FIG. 1(b) shows photographs of the actual Golde hydrogel (top view) and conducting hydrogel electrodes (bottom view) according to a preferred embodiment;

FIG. 1(c) demonstrates the inherent adhesiveness of the hydrogels to various materials including plastic, metal, paraffin, glass, and skin, according to a preferred embodiment;

FIG. 1(d) are photographs of the resulting hydrogels being repaired after cracking showcasing their recoverability thermo-reversibility at the corresponding temperatures, according to a preferred embodiment;

FIG. 2(a) shows SEM imaging of the base hydrogel Golde and the three conducting hydrogels G-TRG, G-MXene and G-PEDOT:PSS, according to a preferred embodiment;

FIG. 2(b) shows corresponding RAMAN profiles of the base hydrogel Golde and the three conducting hydrogels G-TRG, G-MXene and G-PEDOT:PSS, according to a preferred embodiment;

FIG. 2(c) shows corresponding XRD data of the base hydrogel Golde and the three conducting hydrogels G-TRG, G-MXene and G-PEDOT:PSS, according to a preferred embodiment;

FIG. 3(a) shows the swelling ratio profile of the hydrogel variants, according to a preferred embodiment;

FIG. 3(b) shows photographs of the hydrogel being twisted, knotted, stretched and bent after 20 finger bending cycles showing conformability with gloves as well as carrying a mass of ˜35 g of water when attached to a plastic container according to a preferred embodiment;

FIG. 3(c) shows the dog-bone shaped hydrogel samples in tensile testing setup (during failure), according to a preferred embodiment;

FIG. 3(d) shows the stress (kPa) vs elongation (%) curve, where a cross indicates failure, according to a preferred embodiment;

FIG. 3(e) shows the tensile strength (kPa) and elongation (%) of each of the hydrogel electrode variants, according to a preferred embodiment;

FIG. 3(f) shows comparative graph of the elastic modulus values among the hydrogel variants, according to a preferred embodiment, where all the mentioned plots were performed in triplets (n=3);

FIG. 4(a) shows electrochemical impedance spectroscopy: Bode plot demonstrating the average frequency-dependent impedance response of the hydrogel electrode variants with an electrochemical cell setup at a frequency range of 1 Hz-100 kHz and using Phosphate buffer saline 1× with (n=6/variant) according to a preferred embodiment;

FIG. 4(b) shows the Electrical characterization: Skin contact impedance setup as well as plot comparing the commercial clinical grade Ag/AgCl to G-PEDOT:PSS (lowest impedance variant) with (n=3);

FIG. 4(c) shows comparative impedance values at the relevant 100 and 1000 hertz frequencies according to a preferred embodiment;

FIG. 4(d) shows Cyclic Voltammetry (CV) characterization after 3 cycles (top views) and calculated specific capacitance (bottom view) for each hydrogel variant, according to a preferred embodiment;

FIG. 4(e) shows gain response stability from 1 Hz-10 kHz of the fresh standalone hydrogel electrodes compared to electrode samples stored 42 days (see line vs symbols).

FIG. 5(a) shows Electrophysiology recordings: Photograph of ECG acquisition setup according to a preferred embodiment;

FIG. 5(b) shows Electrophysiology recordings: Photographs showing the conformability and stretchability of the self-adherent hydrogel variants according to a preferred embodiment;

FIG. 5(c) shows Electrophysiology recordings: Stacked ECG recording preview of all the electrodes along the corresponding signal-to-noise ratio (dB) according to a preferred embodiment;

FIG. 5(d) shows Electrophysiology recordings: Effect of applying filtering on Ag/AgCl vs G-PEDOT:PSS according to a preferred embodiment;

FIG. 5(e) shows Electrophysiology recordings: G-PEDOT:PSS all-flexible electrode directly self-adhering to the skin using flexible copper tape for interface according to a preferred embodiment;

FIG. 5(f) shows Electrophysiology recordings: ECG recording from the all-flexible electrode setup at rest and during talking according to a preferred embodiment;

FIG. 5(g) shows Electrophysiology recordings: The effect of a motion artifact on the recorded ECG signal on both the Ag/AgCl and G-PEDOT:PSS electrode according to a preferred embodiment;

FIG. 6 shows the effect of storing on the hydrogel according to a preferred embodiment in different humidity levels and sweat on the signal to noise of the ECG recordings; and

FIG. 7 shows the effect of vinegar coating on all-flexible electrode according to a preferred embodiment.

### DETAILED DESCRIPTION OF PREFERRED EMBODIMENTS

A novel hydrogel, method of making and their use as medical electrodes or the like will be described hereinafter. Although the invention is described in terms of specific illustrative embodiments, it is to be understood that the embodiments described herein are by way of example only and that the scope of the invention is not intended to be limited thereby.

The terminology used herein is in accordance with definitions set out below.

As used herein % or wt. % means weight % unless otherwise indicated. When used herein % refers to weight % as compared to the total weight percent of the phase or composition that is being discussed.

By “about”, it is meant that the value of data disclosed herein can vary within a certain range depending on the margin of error of the method or device used to evaluate or measure such data. A margin of ±10% is generally accepted.

The description which follows, and the embodiments described therein are provided by way of illustration of an example of particular embodiments of principles and aspects of the present invention. These examples are provided for the purposes of explanation and not of limitation, of those principles of the invention. In the description that follows, like parts and/or steps are marked throughout the specification and the drawing with the same respective reference numerals.

As aforesaid, it is first disclosed a hydrogel comprising gelatin (or hydrolyzed collagen), chitosan (polysaccharide processed from chitin) and glycerol.

Gelatin, that can be represented by the Formula I below, is a form of hydrolyzed collagen, a protein that constitutes the majority of the human skin composition.

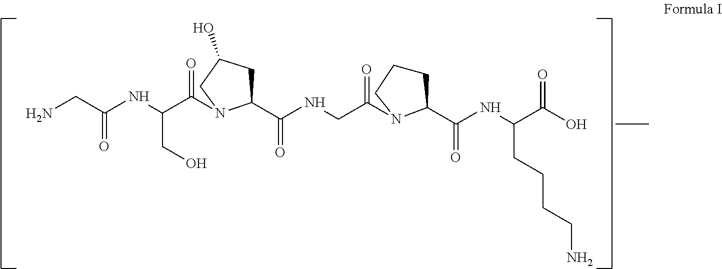

Being both biocompatible and biodegradable, cheap and relatively highly abundant, gelatin has been widely used in several biological applications. For example in wearables, as it possesses a variety of functional groups such as amino (R—NH₂), hydroxyl (R—OH) and carboxyl (R—COOH), it facilitates the formation of polyelectrolyte complexes (PECs) and adherence to the skin [21]. Several studies have hence focused their attention on gelatin-based hydrogel electronics, endowing the hydrogel with electrical conductivity for example by sandwiching it with 2D materials such as polypyrrole, and reduced graphene oxide [17].

Chitosan, that can be represented by the Formula Il below, is the second-most abundant polysaccharide [26].

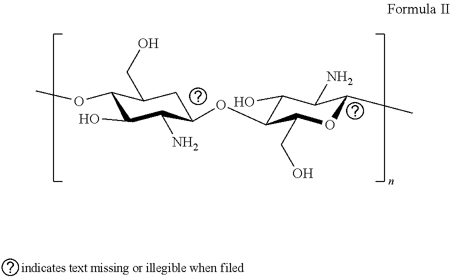

Chitosan is a derivative of chitin, easily obtained from chitin, which is very similar in the chemical structure, and chitosan is bio-adhesive. Similarly, to gelatin, chitosan is also biocompatible and biodegradable, exhibits ionic conductivity, and the ability to form intermolecular interactions such as PECs due to its functional groups [18, 19, 27].

Finally, glycerol, is a triol that is represented by Formula Ill below.

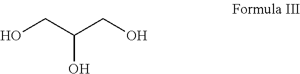

Glycerol is a natural plasticizer employed in several hydrogel electrodes [5, 28]. For example, increasing the ratio of bound water molecules against the free water molecules using its hydroxyl groups has shown to promote hydrogen bonding [28], thereby reducing the gelation time, making the resulting hydrogel more elastic and robust with non-drying and anti-freezing behavior [5, 16, 28].

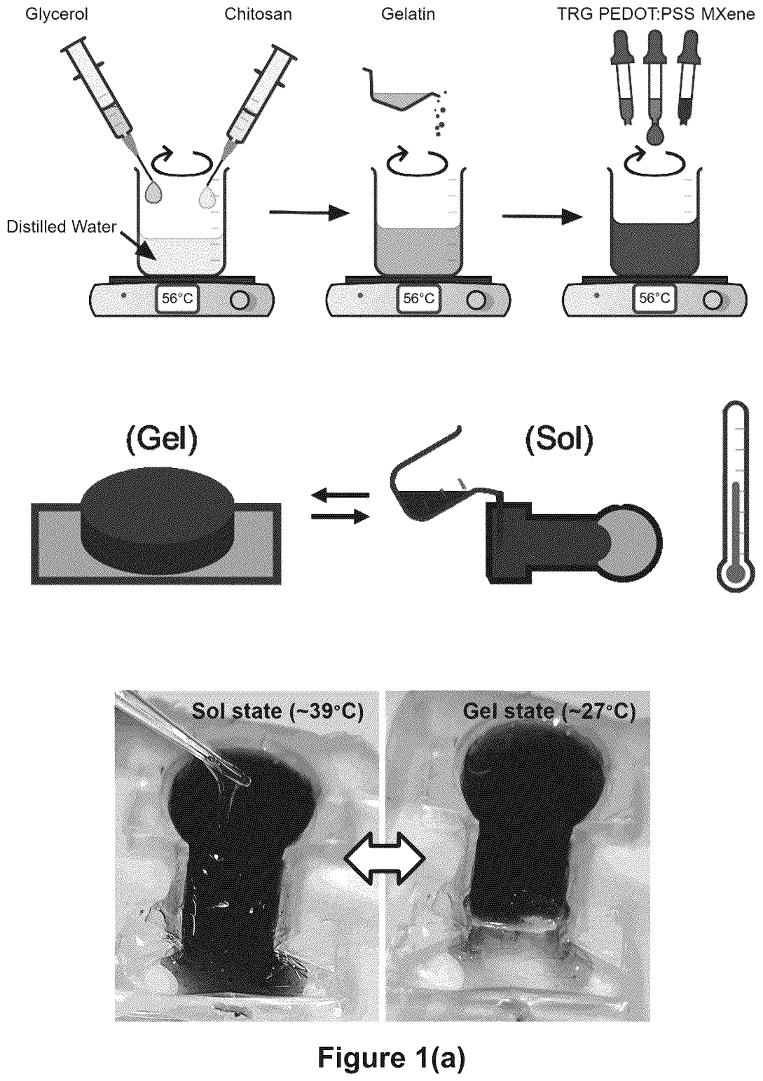

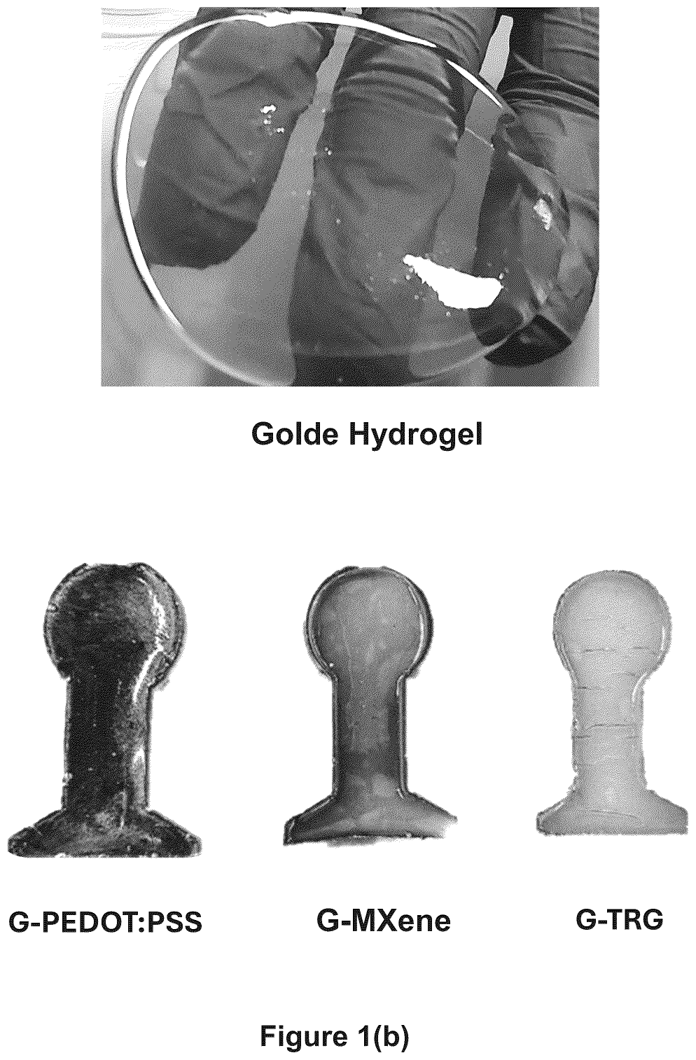

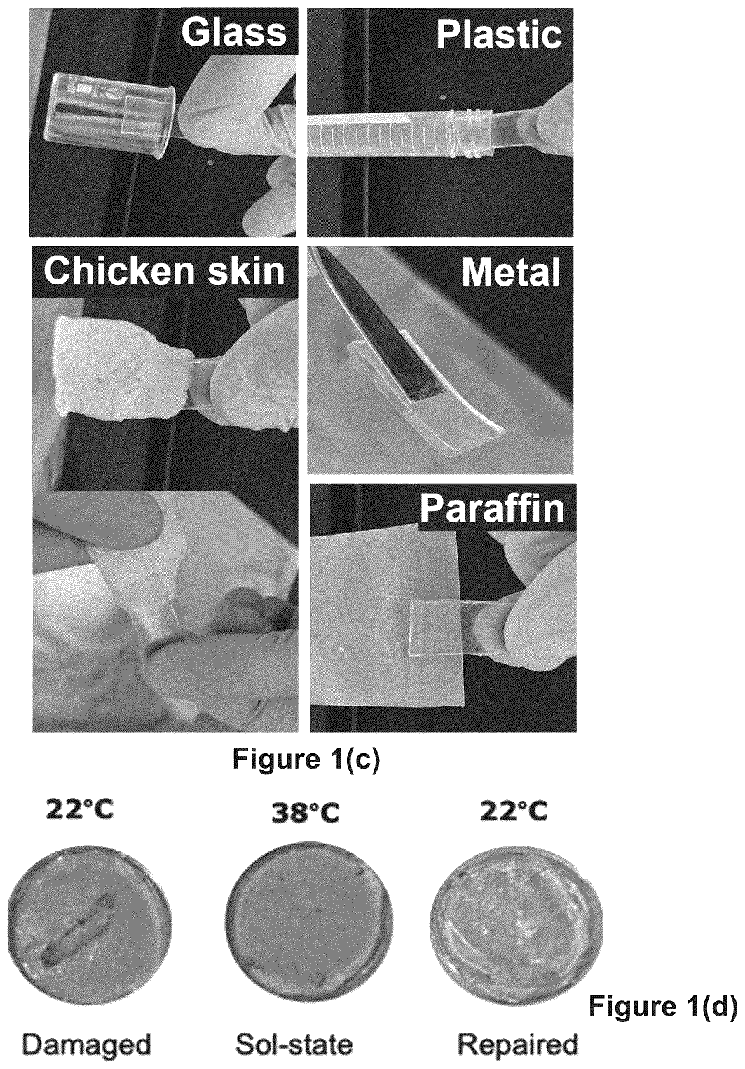

The hydrogels according to the present invention combine gelatin, chitosan and glycerol, benefiting from their synergistic properties through a one-pot fabrication approach as shown in FIG. 1a.

Golde, the resulting hydrogel, depicted in FIG. 1b, bypasses the use of typical crosslinkers such as dimethylsulfoxide (DMSO) or other potentially toxic chemical agents as it relies on a high density of dynamic noncovalent interactions.

It is believed that the combination of chitosan, being a cationic polysaccharide, and gelatin, being an amphoteric protein, at certain pH, enables the formation of electrostatically entangled PECs forming polymeric architectures with relatively high tensile strength for a natural hydrogel.

The hydrogel according to the present invention demonstrates several attractive features: robustness, highly stretchable, inherently adhesive and thermo-reversible. Thermo-reversibility facilitates the repair of the hydrogel upon drying or cracking, which makes it reusable (see FIG. 1a,d). Further, this allows for flexibility and versatility in preparation as it can be re-processed any time or can be used as a viscous ink for 3D printing.

To enhance its conducting properties, three conducting materials have been tested as shown in FIG. 1b, including MXene (Ti3C2Tx), thermally reduced graphene (TRG), and Poly(3,4-ethylenedioxythiophene) polystyrene sulfonate (PEDOT:PSS). The composition of the hydrogel variants was optimized to yield homogenous dispersions of the conductive materials in the hydrogel solution for optimum robustness and conductivity.

All the three resulting hydrogel electrodes are robust, with mechanical strength that outperforms state-of-the-art natural and hybrid-based hydrogels (SI-Table 1 for comparison) and can conform to the skin, with the potential for high-quality ECG recordings. Owing to the inherent adhesiveness of the hydrogel as demonstrated in FIG. 1c in a variety of surfaces including skin, an all-flexible electrode was used to record a Lead I ECG by directly interfacing a healthy 24-year-old male. The present work presents a facile and eco-friendly solution to design sustainable wearable materials for epidermal electronics and electrophysiological recordings with demonstrated superior performance to the commercial electrodes.

### Material Selection

According to a preferred embodiment, three natural hydrogel materials have been combined to create a robust base hydrogel, namely Golde, which refrains from the use of chemical crosslinkers while featuring tissue-like mechanical properties and thermo-reversibility, which is important for synthesis and post-synthesis modification. Typically, mechanical stability and integrity are imparted via crosslinking i.e., formation of chemical or physical bonds between the polymer chains within a hydrogel network. Crosslinking creates a three-dimensional network that prevents the dissolution or disintegration of the hydrogel in an aqueous environment allowing the hydrogel to maintain its shape, strength, withstand mechanical forces and maintain its functionality. Here, chitosan creates a PEC (polyelectrolyte complex) with gelatin, resulting in a tough natural hydrogel with a relatively high tensile strength without the addition of any external crosslinking agent. In addition, no mold or fouling was observed in the hydrogel samples after 3 months of storage in ambient room conditions, possibly attributed to chitosan's antimicrobial properties [26]. Furthermore, the integration of glycerol, is found to improve the hydrogel's elasticity while inhibiting its dehydration by acting as a plasticizer and a humectant

According to a preferred embodiment, the hydrogel as disclosed herein is made electrically conducting and its electrode impedance is lowered by blending the base material (Golde) with a conducting material. Three different conducting materials have been tested: (Poly(3,4-ethylenedioxythiophene) polystyrene sulfonate) also known as PEDOT:PSS, MXenes and thermally reduced graphene (TRG). All three materials were processed from water suspensions and mixed directly with Golde solution (as shown in FIG. 1a,b).

PEDOT:PSS is a conducting polymer that has been widely used in wearable electronics [30], typically interacts with other charged polymers, chitosan herein, creating complexes that may improve the overall hydrogel properties.

MXene, is a composite of a metallic carbonitride, in the present case titanium carbide, in the following notation (Ti3C2Tx) where T represents the surface functional group (—OH, —F, —O). MXene (Ti3C2Tx), exhibits metallic conductivity as well as tunable surface functional groups and negatively charged hydrophilic surface enabling strong interactions with polymer networks such as electrostatic and hydrogen bonds [32].

Thermally reduced graphene, or TRG, features a large surface area and alongside improving the conductivity, its (nano) structure could act as mechanical reinforcement to the hydrogel providing stability and conducting paths through interpolymeric interactions [17].

### Surface Chemistry and Morphology

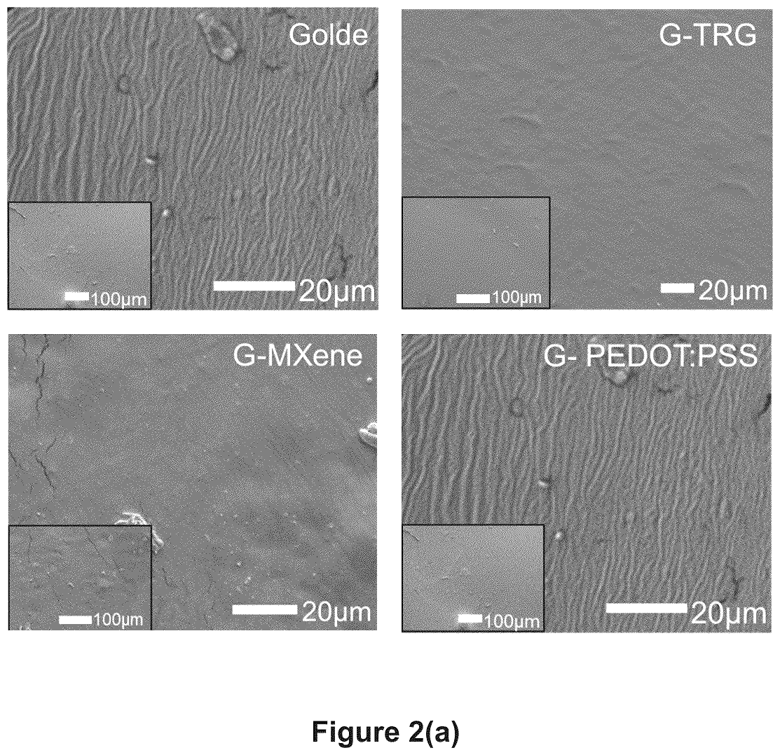

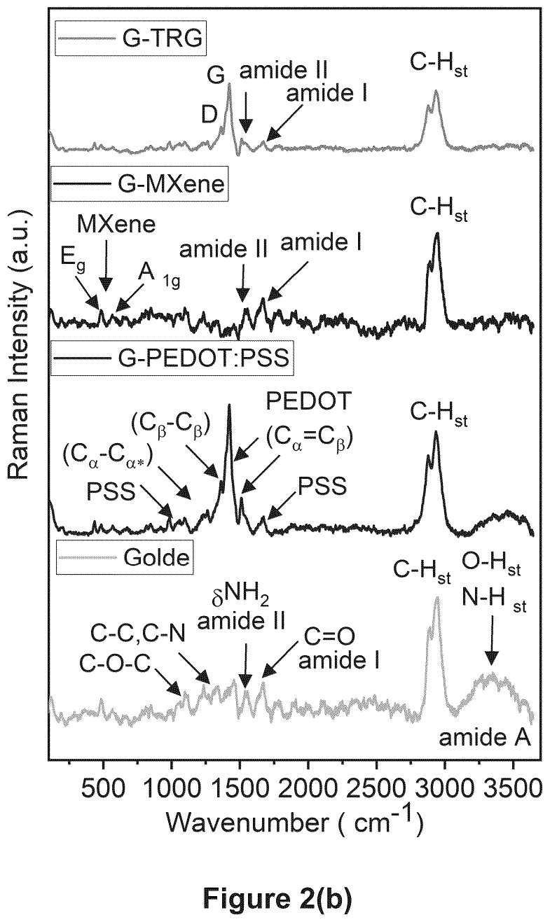

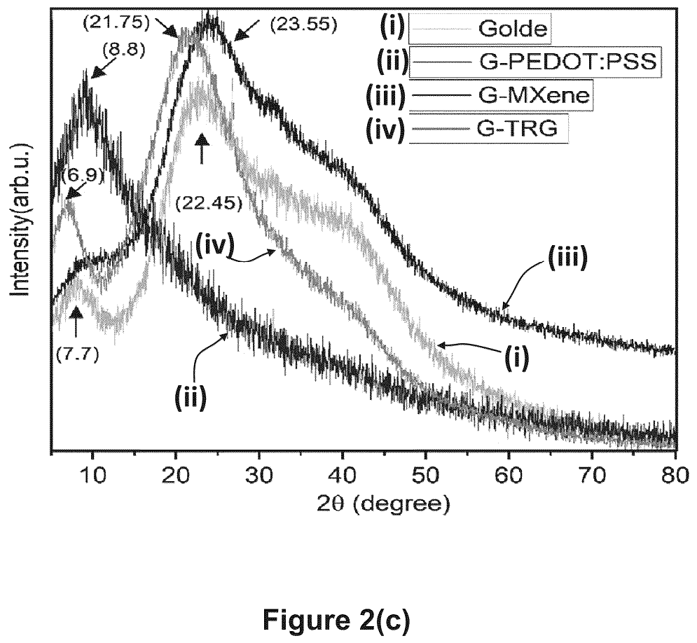

The surface morphology of the different hydrogels is shown in FIG. 2a. Golde is found to have a grain-like morphology at the surface, most likely due to the amorphous architecture into which the biopolymers are intermixed. The addition of MXene and TRG results in a considerably smoother and uniform surface, which is desirable for realizing a conforming and skin-friendly electrode interface. This could be attributed to the fact that 2D carbon materials such as MXene and TRG restrict the polymer movement thus inducing higher resistance before it elongates [32]. Finally, the addition of PEDOT:PSS results in a wrinkle-like surface as a result of the interaction between the biopolymers and the conducting polymer shown previously. All SEM images indicate the uniform integration of the conducting materials with the biopolymers, and the smoothness in all cases is expected to result in higher-quality ECG signals that will be less affected by motion artifacts.

XRD data are shown in FIG. 2(c), indicating that Golde variants possess both crystalline and amorphous nature. FIG. 2b, shows the Raman spectra of Golde, G-PEDOT:PSS, G-MXene and G-TRG samples, for a more detailed Raman spectra analysis refer to FIG. 2(b). Golde sample shows the major Raman bands at 3356 cm⁻¹, 2929 cm⁻¹ and 1656 cm⁻¹ due to O—H and N—H stretching, C—H stretching, C═O stretching (amide I) and NH bending, respectively. After blending with PEDOT-PSS, the major Raman bands of PEDOT are observed 1521 cm⁻¹, 1448 cm⁻¹, 1381 cm⁻¹, and 1267 cm⁻¹, and assigned to the Cα=Cβ asymmetrical, Cα=Cβ symmetrical, Cβ=Cβ stretching, and Cα=Cα′ inter-ring stretching vibrations, respectively. The vibrational modes of PSS are positioned at 1108 cm⁻¹ and 1003 cm⁻¹. G-MXene hybrid sample shows Raman Bands over 500-750 cm⁻¹ are associated with vibrations of C atoms of Ti—C (E1g, E2g and A1g symmetries) and G-TRG sample shows the characteristic D-band and G-band peaks of TRG in the composite are observed at 1348 cm⁻¹ and 1518 cm⁻¹, respectively. The observed characteristic peaks of MXene with low intensity, indicate possibly that MXene is well dispersed in Golde sample without resulting in aggregations. The intensity of D and G peaks is comparatively weak in G-TRG sample indicating that the exfoliated TRG layers are uniformly dispersed in Golde.

### Mechanical Characterization

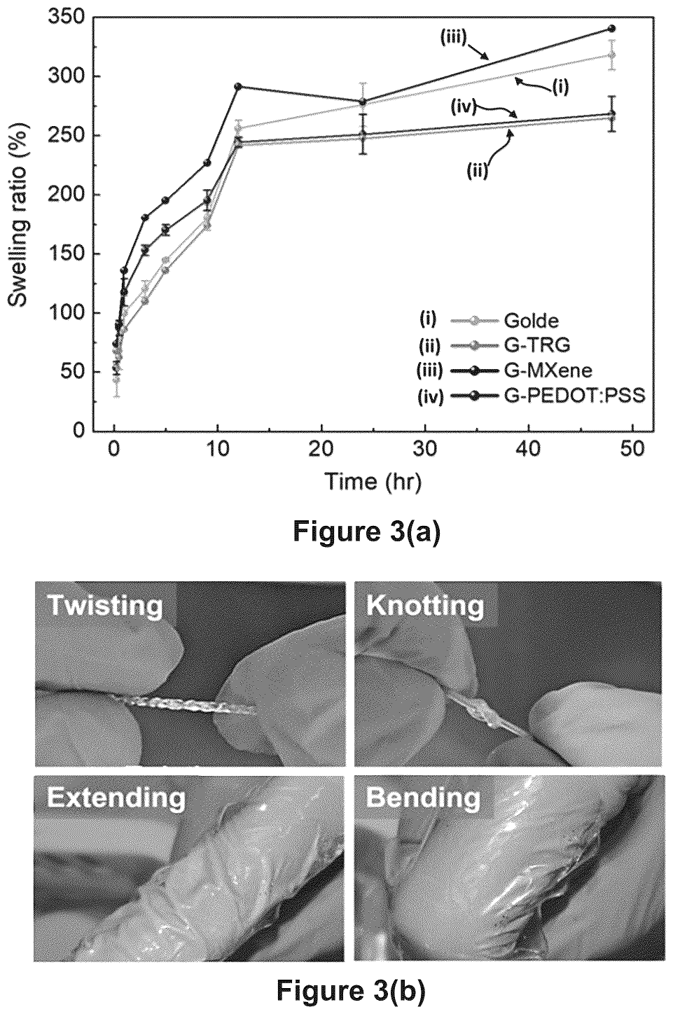

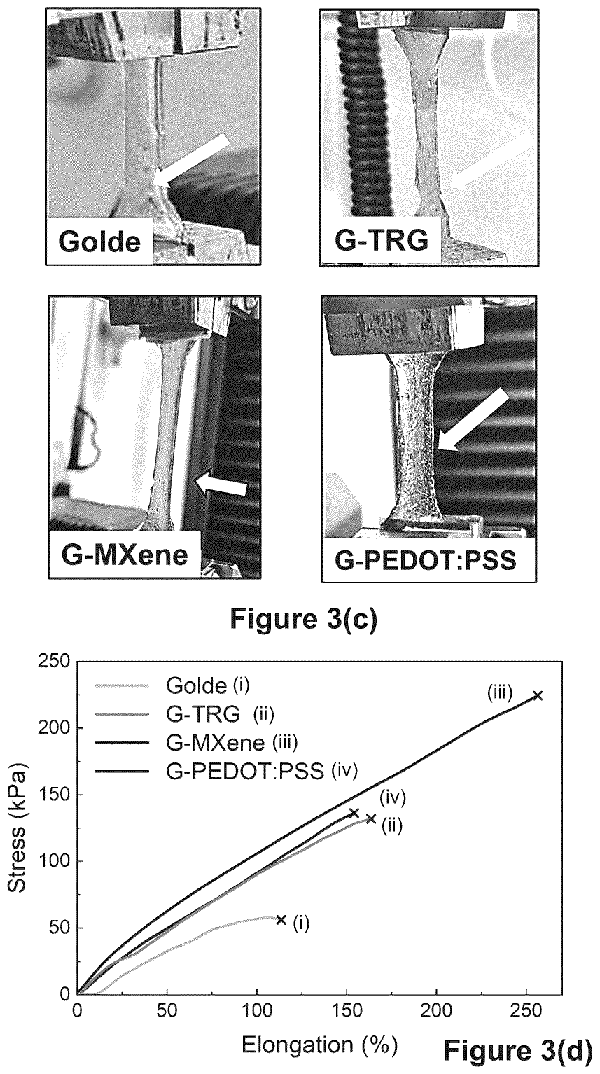

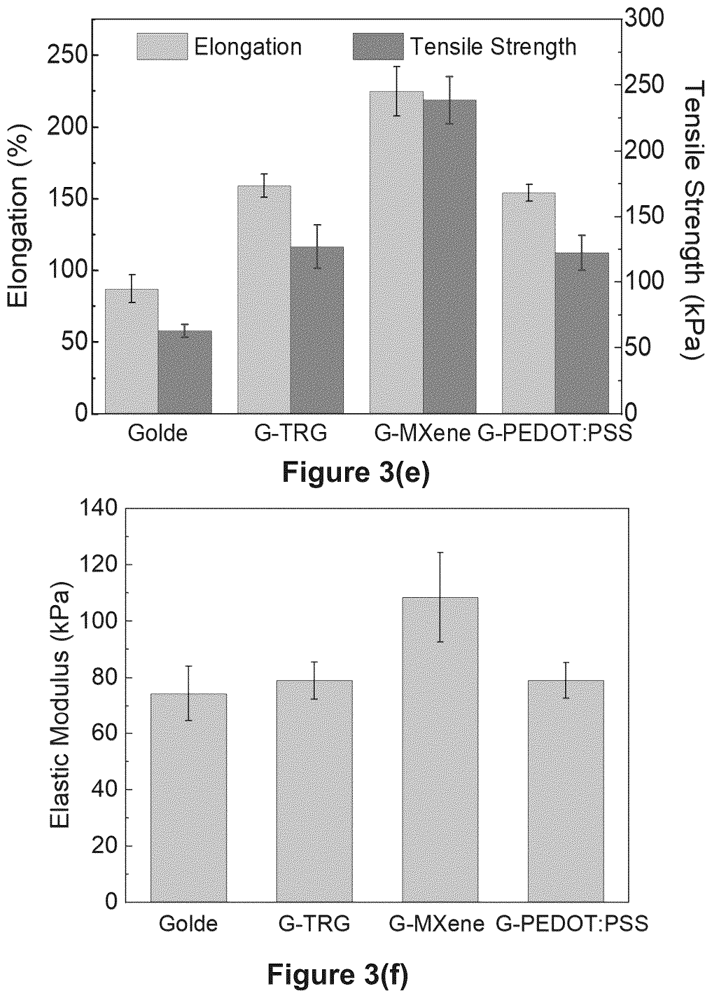

Prior to testing the mechanical performance of the hydrogels, a swelling test (see FIG. 3a) has ben performed to evaluate the water uptake properties of the fabricated hydrogels. Initially, G-MXene and G-PEDOT:PSS experienced a similar trend, having the highest swelling rate. After 12 hours the G-TRG and G-PEDOT:PSS reached saturation and, by 48 hours all four hydrogels show saturation. FIG. 3b shows photographs of the hydrogel being twisted, knotted, stretched, and bent without affecting its shape and robustness as well as its ability to hold a plastic tube with water weighing 35 g. To verify the mechanical properties of the fabricated hydrogels against the human skin and other works in the literature, tensile testing was performed as shown in FIG. 3c and the stress vs elongation curve for each variant is presented in FIG. 3d. The tensile testing of the hydrogels showed that all the variants lay within the elastic modulus that mimics the skin (0.5-500 kPa)[25]. Typically, a higher elastic modulus with stretchable behavior is desirable as it minimizes electrode detachment or displacement during movement, enhancing stability, durability, and signal-to-noise ratio. All variants demonstrate a relatively linear behavior which facilities the estimation of their elastic moduli. Referring to FIGS. 3e and 3f, G-MXene is found to have the highest tensile strength of 225 kPa and an elastic modulus of 87.8 kPa, followed by G-PEDOT:PSS (137 kPa and 88.9 kPa), G-TRG (133 kPa and 81.5 kPa) and finally, Golde, (57 kPa and 50.4 kPa). It is believed that the superiority of G-MXene is mainly due to the ordered intercalated structure of MXene, which introduces more order into the hydrogel polymer matrix and due to its high toughness and abundance of functional groups present on its surface, it simultaneously restricts the movement of the hydrogel polymer chains allowing them to tolerate more stress before fracture occurs in the material. Generally, the proposed conducting hydrogel electrodes supersede the state-of-the-art natural hydrogel electrodes proposed for electrophysiology applications as summarized in Table A disclosed hereinafter.

Table A presents a comparison of the hydrogel electrodes according to the present invention (4 last lines in bold) and other recent relevant works for electrophysiological acquisition in terms of tensile strength and elongation at break with some using chemical or physical cross-linking agent. Other works in the literature are either not tested using the same method or not designed for our application. All materials abbreviations are shown below.

| | | Tensile | Elongation |
|---|---|---|---|
| | Base | Strength | At break |
| Material | Material | (kPa) | (%) |
| PVA, PPy and aramid nanofibers | Synthetic | 9400 | 36 |
| Polymerizable rotaxane hydrogel | Synthetic | 78 | 830 |
| (PR-Gel) | | | |
| PAAm/AAc/Glycerol | Synthetic | 62 | 500 |
| PNIPAM/AAc/Am/LMA | Synthetic | 1010 | 1866 |
| PVA/PAAm/LS/MXene | Synthetic | 630 | 2000 |
| PVA/CMC/PEDOT:PSS | Hybrid | 85 | 74 |
| PAA/ACC/MXene | Hybrid | — | 450 |
| Gel/PPy/rGO | Hybrid | 63 | 250 |
| AgNPs/MXene/GG/Alg-PBA | Hybrid | 25 | 167 |
| Gel/PEDOT:PSS | Natural | 72 | 235 |
| Gel/CS/Glycerol | Natural | 57 | 113 |
| G-PEDOT:PSS | Natural | 137 | 154 |
| G-MXene | Natural | 225 | 256 |
| G-TRG | Natural | 133 | 163 |

Abbreviations: Poly-vinyl alcohol (PVA), Polypyrrole (Ppy), reduced graphene oxide (rGO), Acrylic acid (AA), (PNIPAM), Acrylamide (Am), Polyacrylamide (PAAm), lauryl methacrylate (LMA), Lithium salt (LS), Thermally reduced graphene (TRG), N,N′-methylenediacrylamide (MBA, MBAm), Guar gum (GG) and phenylboronic acid grafted sodium alginate (Alg-PBA), Gelatin (Gel), Chitosan (CS), silver nanoparticles (AgNPs), Carboxymethyl cellulose (CMC), polyethylene-3,4-dioxythiophene: sodium polystyrene sulfonate (PEDOT:PSS).

### Electrochemical Characterization

As shown in equation 1, the reactance Xc is inversely proportional to the frequency (f) of the current passing through the material with capacitance (C).

$$Xc = 1 / [ 2 π fC ] \qquad Equation 1$$

Another contributing element is the frequency independent real element (resistance, R) which is a scalar. As shown by equation 2, the total impedance (Ztotal) is the vector sum of its resistance and reactance, as frequency decreases the reactance contribution becomes the dominating element [34]. Therefore at the highest frequencies the impedance is stable.

$${Z}_{total} = R + jXc \qquad Equation 2$$

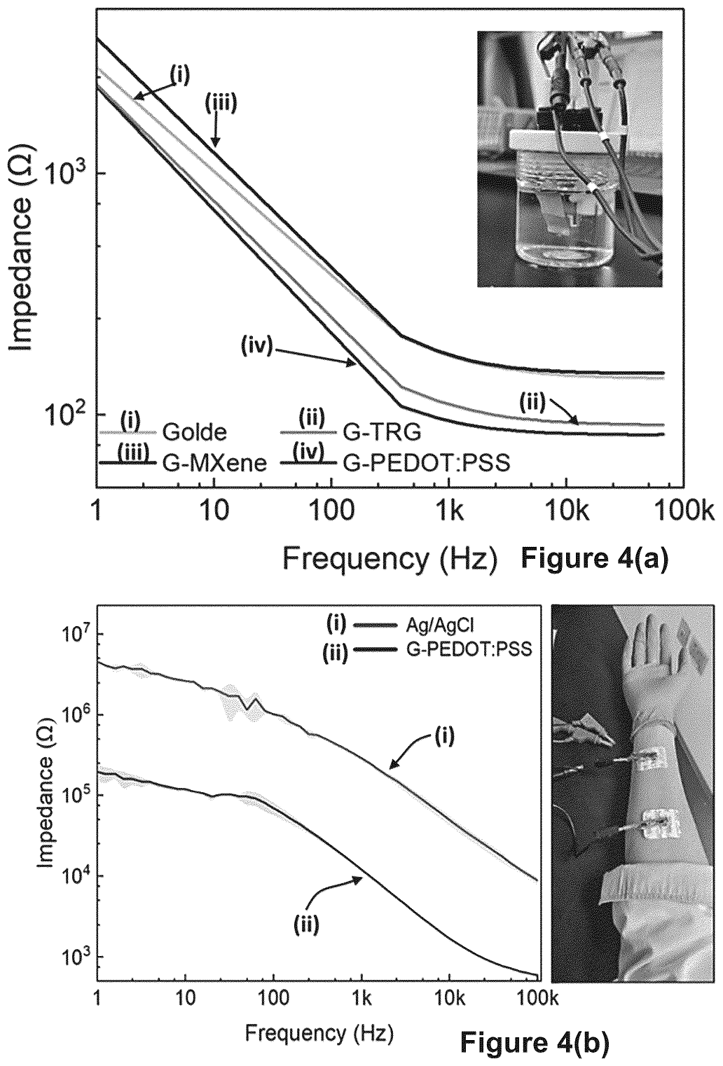

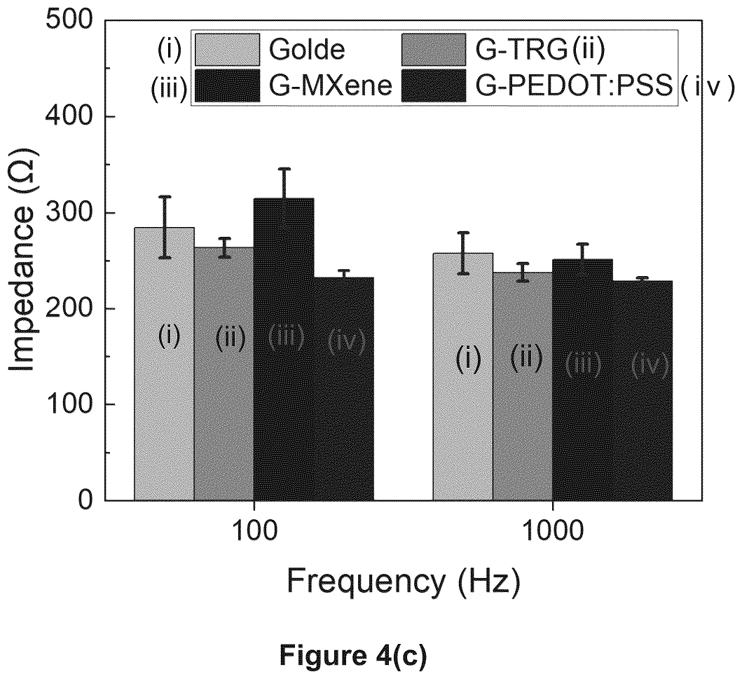

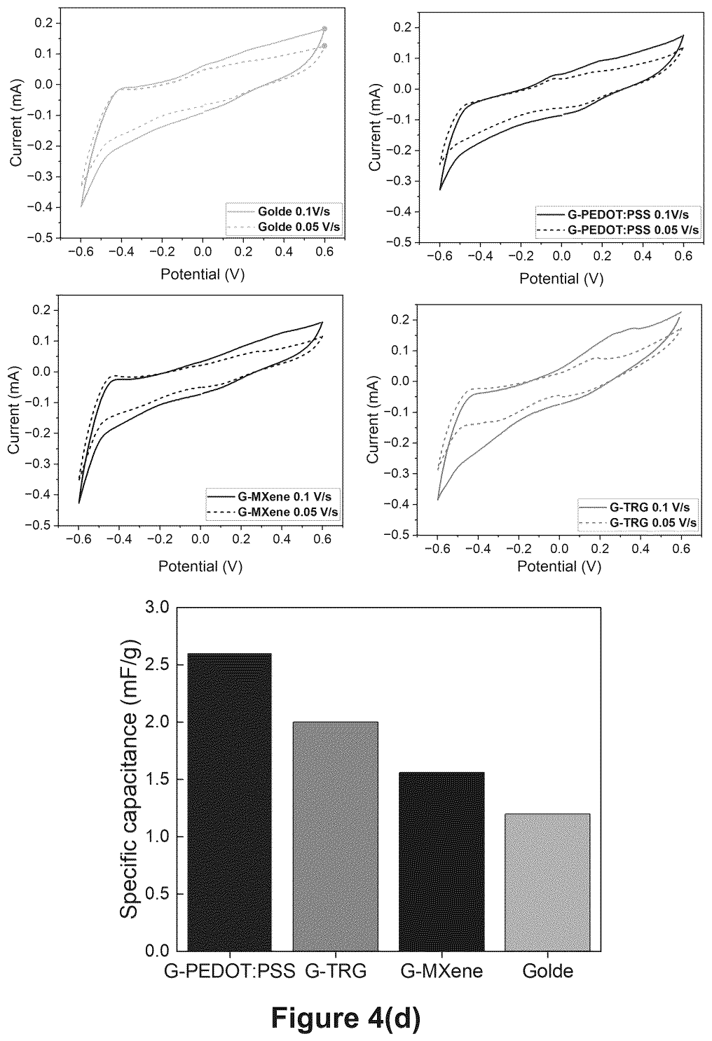

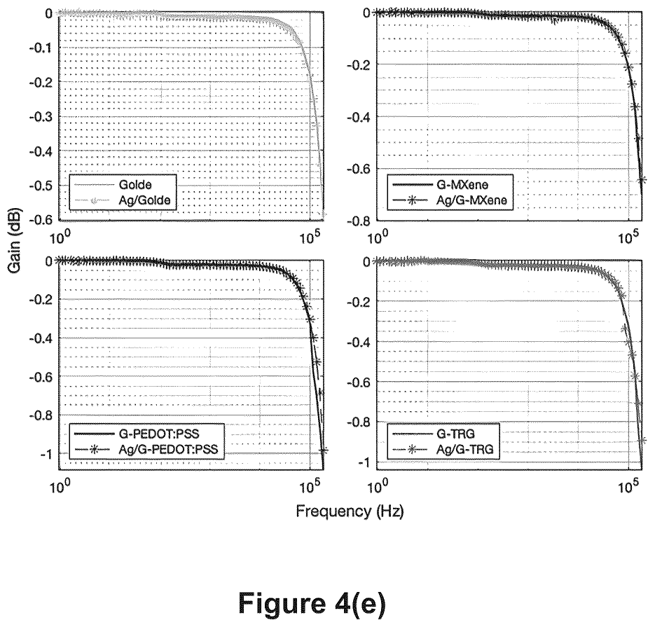

FIG. 4a shows a comparative bode plot of the impedance of the four hydrogel variants over a wide range of frequencies from 1 Hz-100 kHz. It should be noted that the curves shown in FIG. 4a are the average curves of 3 samples/variant where each is measured 3 times (i.e., 9 readings/variant). As expected, the hydrogel electrodes' impedance demonstrated a clear transition from resistive to capacitive regime at lower frequencies, due to the presence of a mixture of charge carriers (see FIGS. 4(a) and 4(c)). Overall, the conducting hydrogels exhibit higher capacitance and lower resistance (hence improved conductivity) compared to the base material, highlighting the uniform distribution of the conducting materials within the hydrogel base material and the favorable interactions with the (bio)polymeric network. The impedance values at the relevant frequencies for electrophysiological sensing are shown for easier comparison in FIG. 4(c), (i.e., 100 Hz, where a capacitive behavior is expected and at 1 kHz, where the dominating impedance behavior is resistive). A skin contact impedance was performed as shown in FIG. 4b, directly comparing the commercial Ag/AgCl electrodes with the G-PEDOT:PSS variant, showing a significant (more than 1 order of magnitude) decrease in the skin contact impedance in the case of the gel electrode. Overall, the low impedance of the hydrogel electrodes allows for efficient charge transfer between the electrode and the biological tissues, resulting in improved signal quality and reduced noise during electrophysiological recordings.

The EIS (Electrochemical impedance spectroscopy) and CV (Cyclic Voltammetry) curves of the electrodes are shown in FIGS. 4a and 4d. CVs were performed at two different scan rates (0.1, 0.05 mV/s) and a potential range of-0.6 to 0.6 V. The CV analysis showed stable, reversible, and well-defined curves [36]. In all samples, as the scan rate increased, the current and area under the curve of the electrodes increased, with little change in the shape of the CV, indicating the effective ion transport processes. This effect was more pronounced on the G-TRG hydrogel electrode. The calculated specific capacitance area of each hydrogel variant is summarized in FIG. 4d, with the G-PEDOT:PSS exhibiting the highest, followed by the G-TRG and G-MXene. The capacitive behavior of the hydrogel electrodes is sought to increase SNR (signal-to-noise ratio) [7, 8] as they are not expected to undergo electrochemical reactions at the electrode-tissue interface that could interfere with accurate recordings.

The stability of the hydrogel electrodes (in terms of their electronic properties) was evaluated by monitoring the change in gain over frequency (FIG. 4(e)) for all 4 electrode variants after 42 days of storage in ambient room conditions. As it can be seen, the electrode properties remain unchanged (see lines—fresh gels vs symbols-old gels in each electrode variant), highlighting their reusability (owing to the thermo-reversibility) and long-term storage capability, which is not possible with Ag/AgCl, which can neither be re-used nor stored for long-term as they tend to dry and degrade. Overall, the results indicate that the conducting material-infused hydrogel electrodes exhibit good electrical performance, superior to that of the base material alone for the intended application, with no apparent differences observed between the different variants.

### ECG Recordings: Commercial Ag/AgCl Versus Conducting Hydrogel Electrodes

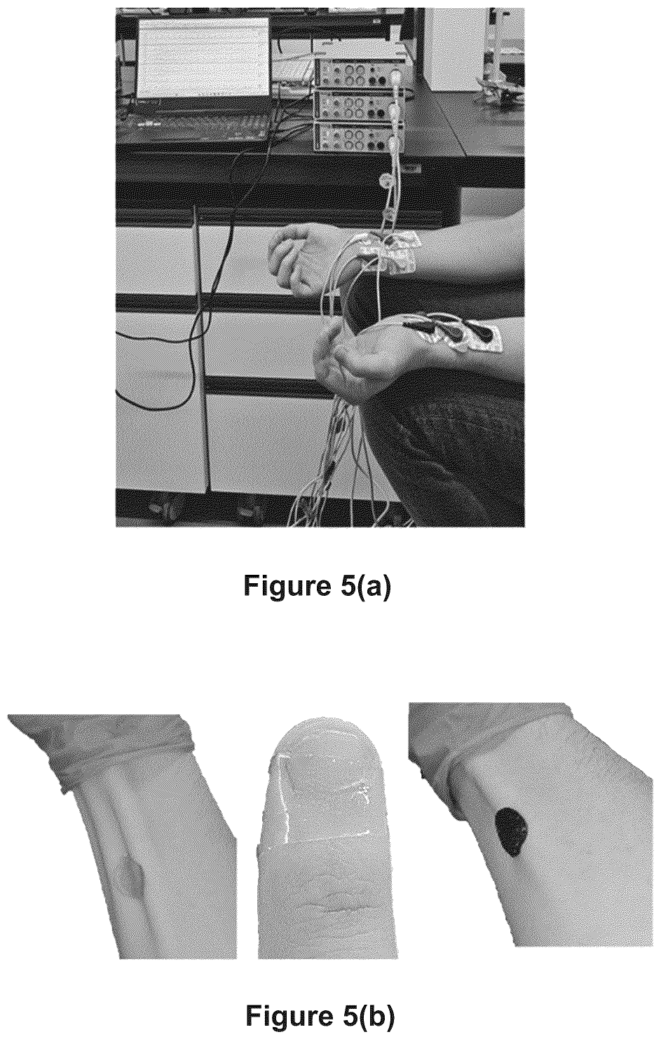

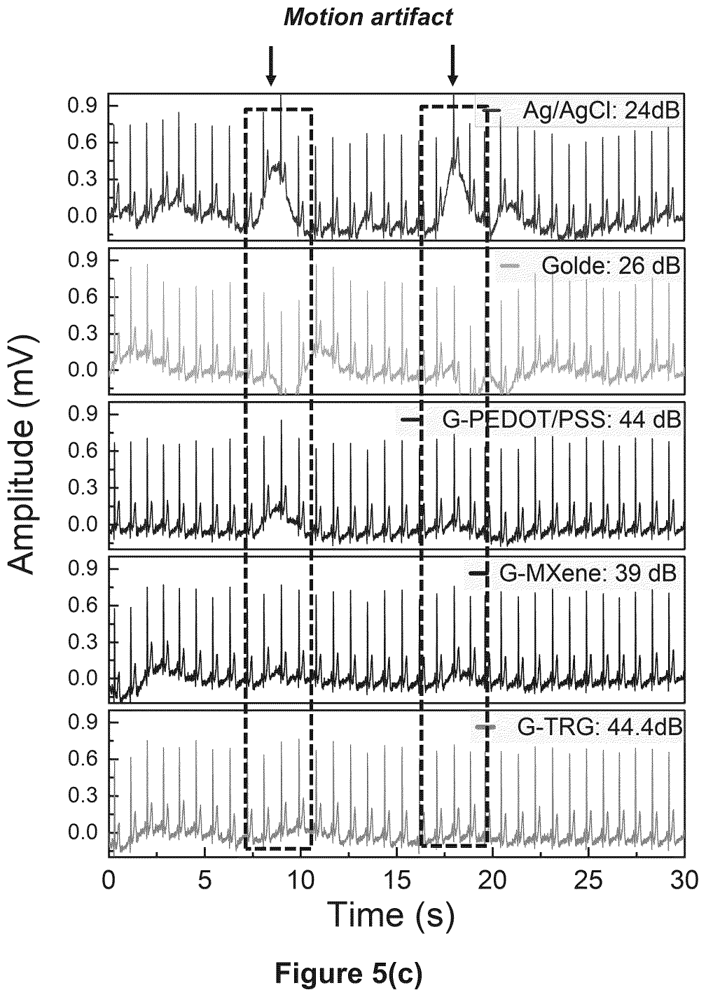

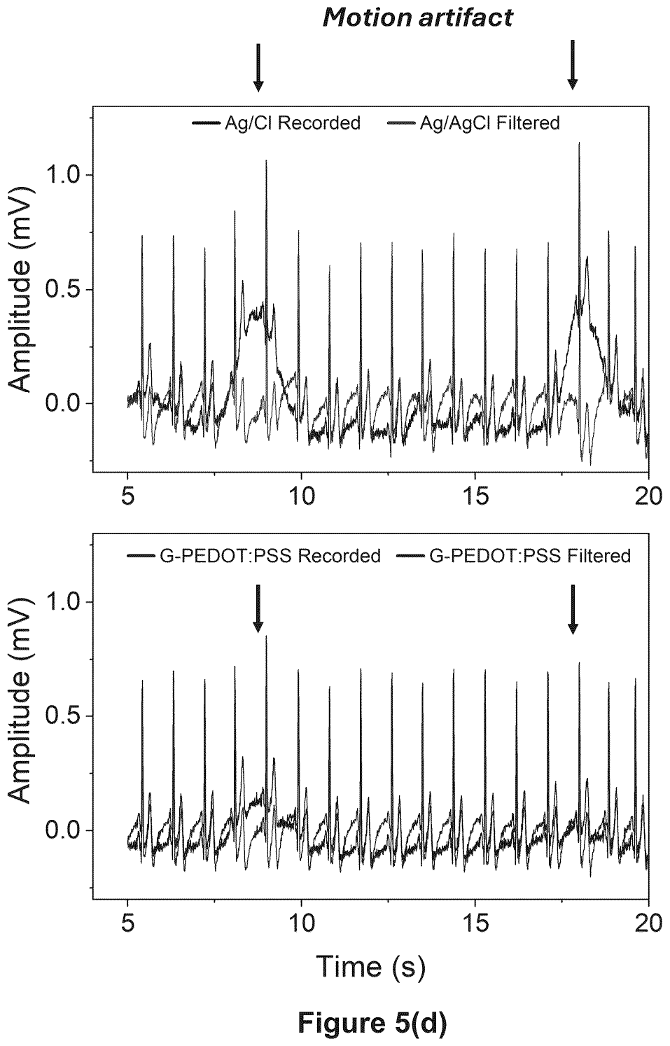

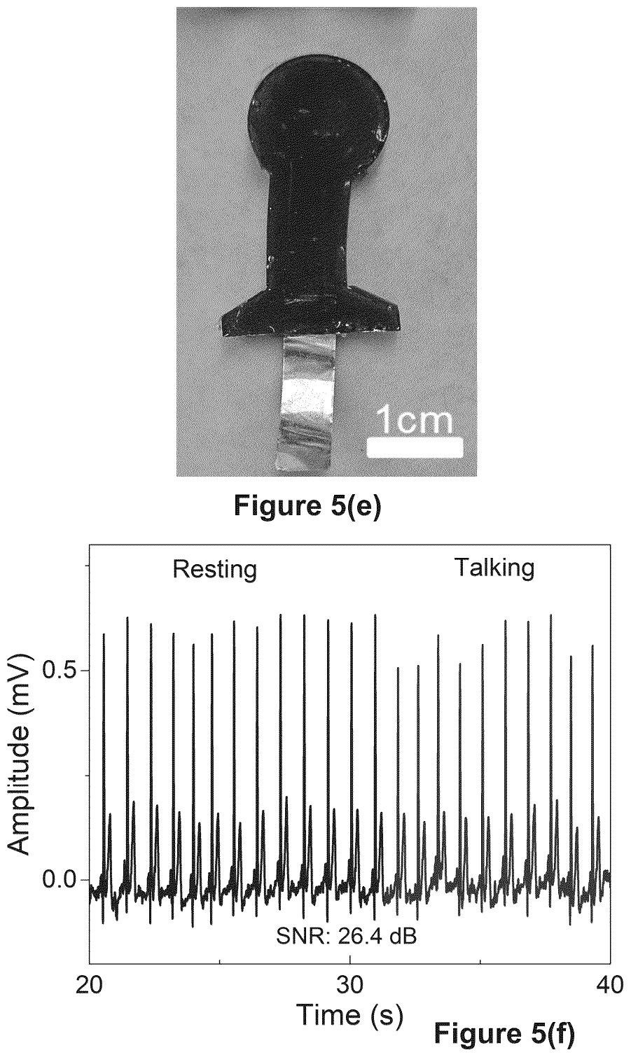

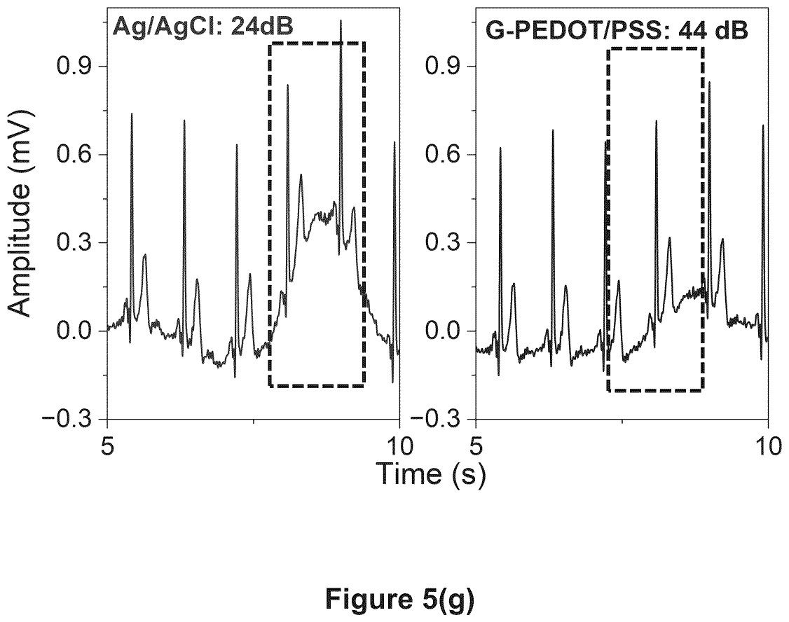

The hydrogel electrodes' low electrode impedance (see FIG. 4a), superior mechanical compliance (see FIG. 3 and Table A), and inherent adhesiveness demonstrated their suitability for acquiring ECG signals as efficient electrolyte-retention elements. Using a lead I ECG placement, four sets of the same hydrogel electrodes and a set of Ag/AgCl electrodes were used to obtain the ECG signals from two consenting adult males. The ECG signals were recorded with a setup that was comprised of a PowerLab™ with a bio-amplifier and a computer with LabChart™ software (see FIG. 5a). The hydrogel electrode variants were used separately for one minute while at rest. The same electrodes were used on both volunteers. The resulting ECG signal features were visible, stable and reproducible across the volunteers. Following, all hydrogel electrodes and the commercial Ag/AgCl electrode were used simultaneously for 30 minutes to assess their performance at more extended recording periods. The G-hydrogels were attached to the hand of a representative participant (FIG. 5a,b), in the same way (and locations) as the commercial Ag/AgCl ECG electrodes for comparison. Remarkably, the attachment site of the hydrogel electrodes exhibited no apparent skin irritation following the 30-minute ECG recording session. In contrast, in the case of Ag/AgCl electrodes, it has been noted a slight skin redness and the occasional hair removal of the hair of the arms possibly due to the strong adhesiveness of the adhesive used. Moreover, the hydrogel's pH was tested using litmus paper and found to be in the 6-7 range which is in line with that of the skin. According to the tensile data, the hydrogel electrodes are at the modulus range of the skin, allowing for more than 30% elongation, which is the minimum required for over-the-skin hydrogel patches for wearable applications to conform and be unaffected by skin movement as opposed to the rigid Ag/AgCl electrodes. Indeed, the G-hydrogels according to the present invention adhered well to the skin, possibly reducing contact imperfections caused by human activity (see almost undisturbed ECG compared to the Ag/Ag/Cl one, after hand bending motion artefact as indicated by the black arrows in FIG. 5c).

FIG. 5c illustrates the time-domain representation of ECG signals recorded simultaneously with the four hydrogel electrode variants and the commercial Ag/AgCl electrode. Distinguished and minimized fluctuated P, QRS, and T waves are clearly identified and visible in the ECG waveforms for all electrode variants. Additionally, the ECG signals from all electrode variants are very similar. The comparison of the SNRs of the signals acquired by the different electrodes offers a clear perspective on the relative performance of those electrodes. The SNR was calculated by extracting the signal power from the 0.5-40 Hz band and the noise power from 0-0.49 Hz and 41-500 Hz. Superior signal clarity was demonstrated by the Ag/G-TRG electrode and Ag/G-PEDOT:PSS electrode, with values ˜44 dB. The Ag/G-MXene electrode exhibited an SNR of 39.2 dB, while the Ag/Golde and Ag/AgCl electrodes exhibited the lowest values for SNR, with 25.8 dB and 24.2 dB, respectively. Interestingly, the base hydrogel material exhibited slightly higher SNR compared to the commercial electrode.

FIG. 5d shows the effect of filtering on the recorded raw data for a representative G-hydrogel electrode and the commercial one. The upper half depicts the raw recorded ECG signals while the lower half depicts the signals after further digital processing of the Ag/AgCl and the G-PEDOT:PSS (as a representative of the hydrogel variants) using MATLAB, bandpass filter modified to a range of 0.5-40 Hz. In the G-PEDOT:PSS electrode recording, the R-peak values are almost identical after cleaning the signals, while the P-, Q-, S- and T-waves differ only slightly, suggesting the superior ability of the hydrogel electrode to capture the signals with minimal noise interference. This is also evidenced in the data recorded with motion artefact, where, compared to Ag/AgCl, the hydrogels showed significantly reduced susceptibility to interference. Overall, the G-hydrogels acquired high-quality ECG signals after 30 mins of continuous use with higher SNRs and almost unaffected behavior during motion artifact.

### Stability of the Hydrogels

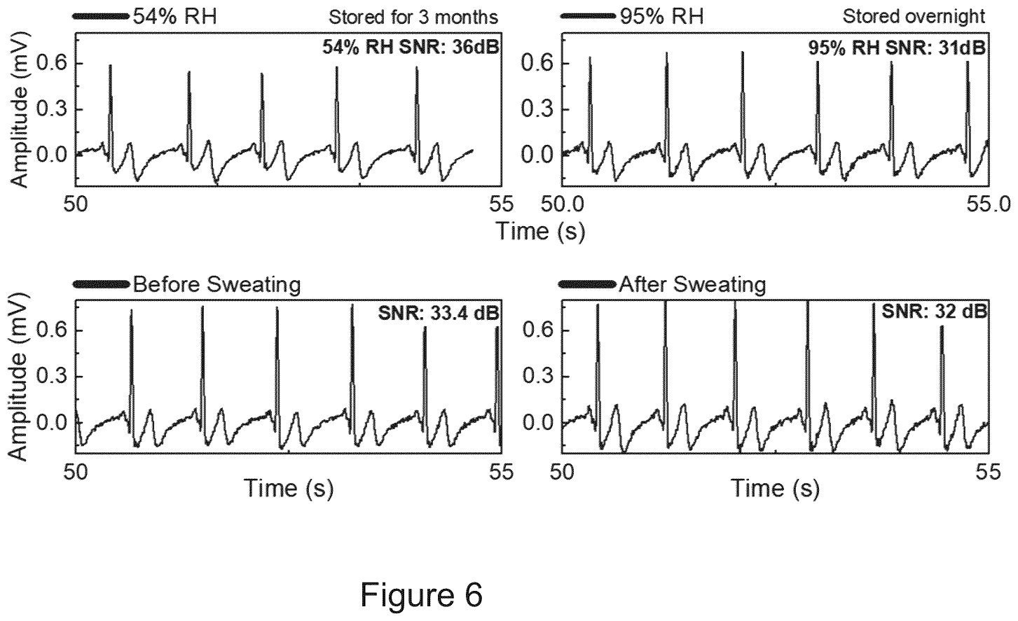

The long-term stability of the electrodes was tested after 3 months of storage under ambient room conditions with a decrease in SNR of 18.2% compared to a freshly prepared sample (see FIG. 6) still outperforming the commercial one. Due to the hygroscopic nature of hydrogels, we also investigated the effect of humidity on the hydrogel electrodes signal acquisition by enclosing the G-PEDOT:PSS electrodes in a room at the extreme condition of 95% relative humidity (RH) overnight, followed by a 10-minute-long ECG recording. The performance of the G-PEDOT:PSS hydrogel showed a 13.3% decrease in the SNR (see FIG. 6). Furthermore, the effect of sweat on the performance of the hydrogel was tested by performing ECG in sweat conditions showing only a negligible decrease of 0.04% in the SNR.

### All-Flexible Self-Adhesive Hydrogel for ECG

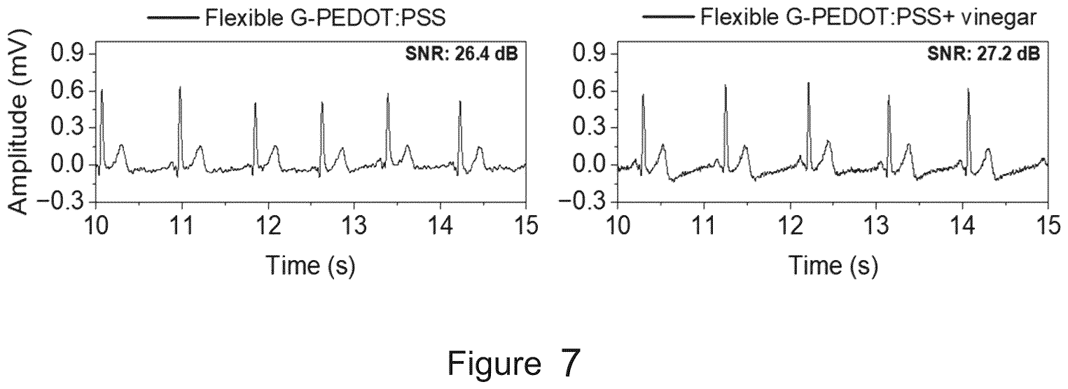

To further evaluate the “standalone” performance and skin-adhering properties of the hydrogel electrodes, G-PEDOT:PSS electrode was fabricated as a proof of concept on an all-flexible electrode setup using copper tape for the connection, as shown in FIG. 5e as a simpler, cheaper and more eco- and skin-friendly solution to the commercially available one. The electrode design was based on a standard commercial clinical grade Ag/AgCl electrode (SkinTact™) with a 1.75 cm diameter circle which would act as the active sensing area. Moreover, since the ECG signal direction is radially symmetric, the circular shape is preferred [37]. Due to the contrasting nature of hydrogels and metals, a 1×1.3 cm rectangular area of the all-flexible electrode was used for interfacing the hydrogel with the copper tape, this area was covered and was not contributing to the signal acquisition (e.a. not touching the skin). Since all variants of the hydrogel according to the present invention exhibit similarly high performance, the choice of G-PEDOT:PSS variant is based on the commercial availability of PEDOT-PSS, simplicity in processing and lower cost compared to MXenes and TRG. The hydrogel variant used herein was stored for 42 days before rehydrating it and using it to develop the all-flexible electrode. FIG. 5f shows the ECG recording on a consenting 24-year-old male during resting and talking. The resulting ECG signal was clear and visible and comparable to the commercial electrode. During resting or baseline conditions, the ECG signal exhibited a regular pattern with the characteristic waves and intervals, while during slight activity (talking), we can see a change in the recorded ECG patterns, as expected, compared to baseline possibly attributed to alterations in heart rate and autonomic nervous system activity. The flexible electrode was also found to be less susceptible to motion artefact interference compared to the commercial electrode (FIG. 5g). Furthermore, its calculated SNR was found to be 26.4 dB (still higher than that of the Ag/AgCl and Golde) which was lower than that recorded for the same variant (˜44 dB) using the Ag conventional setup and adhesive G-PEDOT:PSS. It is believed that this could be attributed to a number of factors related to the setup (copper tape) used to interface the electrode with the measurement unit which necessitates further optimization and/or to the adhesiveness of the hydrogel which could also be improved. Nevertheless, the G-PEDOT:PSS flexible electrode was accurate in recording the ECG at rest and identifying changes during slight activity (talking), exhibiting slightly higher SNR than the commercial electrode and was found less susceptible to interference compared to Ag/AgCl. We note that a small improvement in the adhesion of the all-flexible hydrogel was observed after a small drop of a commercial vinegar (˜30 μL) which was reflected in the SNR value that was improved from 26.4 dB to 27.2 dB (see FIG. 7). This could be attributed to an improved interaction between the hydrogel's functional groups and the skin, by lowering the pH.

### CONCLUSIONS

It has been developed a self-adhering, and reusable conducting hydrogel that does not require any crosslinker, using a simple one-pot synthesis method. The hydrogel is composed of a natural biopolymer blend (gelatin, chitosan and glycerol) and is infused with three types of electrically conducting materials: PEDOT:PSS, MXene and TRG. The resulting hydrogel electrodes namely Golde (biopolymer blend), G-PEDOT:PSS, G-MXene and G-TRG were characterized according to their structure, surface composition, and mechanical and electrical performance.

The mechanical properties of the hydrogels match those of the skin (0.5-500 kPa) [25] while exhibiting stretchability and robustness, outperforming similar natural hydrogels in literature. Morphological and structural analysis revealed favorable interactions between the conducting materials and the biopolymer network, further corroborated by the improvement in the mechanical and electrical properties of the conducting material-infused hydrogels compared to the base material, Golde.

The hydrogel variants as disclosed herein were used as an electrode alternative to commercial Ag/AgCl for electrophysiology recording, herein ECG. The recorded SNR of the hydrogel electrodes was considerably higher than that of the commercial ones and showed no skin irritation and pain-free removal after 30 mins of ECG recording. Furthermore, benefitting from the inherent adhesiveness of the hydrogel an all-flexible electrode setup was developed using copper tape for the connection. The ECG recordings showed less noise and motion artifact susceptibility compared to the commercially available Ag/AgCl. Moreover, although other variants might have shown better performance in some aspects, G-PEDOT:PSS variant was overall the best taking into account the cost and ease in processability alongside SNR performance and skin friendliness.

The hydrogels as disclosed herein outperforms the commercial one; hence, it could be easily integrated into wearable setups, possibly enhanced with the augmentation of Al for advanced health monitoring and analytics [38].

Although herein demonstrated for ECG, it Is believed that other types of measurements would show similarly superior performance. Further, the presence of glycerol in the base material and the low sol-gel (glassy state) temperature i.e., thermo-reversibility of the hydrogel, give the hydrogel a thixotropic nature rendering it a potentially good candidate for extrusion-based hydrogel 3D printing or other applications.

### Materials

Gelatin (Bovine Skin, Type B, G9391, USA), Chitosan (high molecular weight, deacetylated chitin, 419419, Ireland), Acetic acid 100% and Glycerol 99%, Ethylene glycol (EG), 4-Dodecylbenzenesulfonic acid (DBSA) and 3-glycidyloxypropyl) trimethoxysilane (GOPS) were obtained from Sigma-Aldrich Co. Aqueous PEDOT/PSS dispersion (CLEVIOS PH 1000™, 1L, Germany) from Heraeus GmbH & Co. MAX phase (Ti₃C₂Al powder, ≤40 μm, Ukraine) was obtained from Carbon-Ukraine Ltd. Graphite powder (Sigma-Aldrich, 10 mesh), sulfuric acid (Sigma-Aldrich, ACS reagent, 95.0-98.0%), hydrochloric acid (Sigma-Aldrich, ACS reagent, 37%), Lithium Fluoride salt (Sigma-Aldrich), potassium permanganate (Fischer Scientific, C99%), hydrogen peroxide (Sigma-Aldrich, 30 wt % in H2O) were used. Graphite oxide was prepared from natural graphite by using improved synthesis proposed by Tour et al.

### Chitosan Solution Preparation

Different chitosan (CS) solutions have been prepared as indicated in Table 1 below:

TABLE 1
Chitosan (CS) solution for total solvent 10 mL water

| | Acetic acid (99%) | |
|---|---|---|
| Samples | (v/v %) | CS solution wt % |
| Golde Hydrogel | 1% | 0.5 |
| CS+ | 1% | 1 |
| CS++ | 1% | 1.5 |
| CS+++ | 10% | 5 |

More preferably, the chitosan solution of 0.5 wt % was prepared by measuring 10 mL of distilled water at 85° C. into a 50 mL borosilicate beaker stirred at 250 rpm. 0.05 g of the chitosan powder is added, and 100 μL of Acetic acid (dropwise) is added upon dispersion into the solvent. The solution is stirred for preferably 2 hours until the chitosan dissolves completely. The solution is preferably transparent and thick without visible fibers.

### Preparation of the Hydrogel Solutions

Different hydrogel solutions have been prepared as indicated in Tables 2 and 3 below:

TABLE 2
Different preparations of hydrogels

| | Intial | Volume of | | | | | |
|---|---|---|---|---|---|---|---|
| | Volume | chitosan | Volume | Mass | Volume | | Ratio of |
| | of | solution | of | of | of | Total | Water |
| | Water | (0.5 wt %) | glycerol | gelatin | gelatin | volume | to Total |
| Scenario | (mL) | (mL) | (mL) | (g) | (mL) | (mL) | Solution |
| Initial | 5 | 1.5 | 1.5 | 1.75 | 1.35 | 9.35 | 53.48% |
| water−− | | | | | | | |
| Intial | 7.5 | 1.5 | 1.5 | 1.75 | 1.35 | 11.85 | 63.29% |
| water− | | | | | | | |
| Golde | 12 | 3 | 3 | 3.75 | 2.88 | 20.88 | 57.47% |
| Hydrogel | | | | | | | |
| Intial | 20 | 3 | 3 | 3.75 | 2.88 | 28.88 | 69.25% |
| water+ | | | | | | | |
| Intial | 12 | 1.5 | 1.5 | 1.75 | 1.35 | 16.35 | 73.39% |
| water++ | | | | | | | |
| Intial | 20 | 1.5 | 1.5 | 1.75 | 1.35 | 24.35 | 82.14% |
| water+++ | | | | | | | |

Gelatin density ~ 1.3 g/mL

TABLE 3
Different hydrogel compositions
Hydrogel Composition

| Material | Proportions | | | | | | |
|---|---|---|---|---|---|---|---|
| Gelatin | 1 | 1.5 | 1.75 | 1 | 3 | 2.5 | 1 |
| Chitosan (0.5 | 1 | 1 | 1 | 2 | 2 | 1 | 1 |
| wt % solution) | | | | | | | |
| Glycerol | 1 | 1 | 1 | 3 | 1 | 1 | 3 |
| Ratio | 1:1:1 | 1.5:1:1 | 1.75:1:1 | 1:2:3 | 3:2:1 | 2.5:1:1 | 1:1:3 |

More preferably, following a simple one-pot process, a volume of 12 mL of distilled water was added to a 50 ml beaker and stirred at 160 rpm at no more than 55° C. In this order, 3 mL of glycerol were added followed by 3 mL of the prepared chitosan solution and wait until the solution is mixed thoroughly (not more than 5 min). Afterwards, slowly increase the stirring speed to 350 rpm and add 3.75 g of the gelatin powder to avoid aggregation. After 20 minutes the solution becomes homogenous and transparent with golden/amber color. To prepare larger amounts of the Golde hydrogel solution, all the ratios must be consistent.

### PEDOT:PSS Solution Preparation

5% ethylene glycol (EG), 0.5% 4-Dodecylbenzenesulfonic acid (DBSA), 1% 3-glycidyloxypropyl)trimethoxysilane (GOPS) added to the aqueous PEDOT/PSS dispersion. Using a digital scale in this order EG, DBSA, GOPS were added to the falcon tube. After adding each additive, the solution was sonicated for 5 minutes (I.e., EG, sonicate, DBSA, etc.) Finally, using a syringe filter of 0.8 μm pore size to complete the final mixture that would be used. The GOPS was added to the PEDOT:PSS mix later (just before mixing it with the hydrogel solution) or not at all to avoid having clumps that prevents the PEDOT:PSS from uniformly dispersing within the hydrogel solution.

### MXene Solution Preparation

The MAX phase substance, Ti3C2Al powder, with a size of up to 40 μm, was utilized to produce MXene (Ti3C2Tx) following a carefully refined minimally intensive layer delamination (MILD) process. This technique involves the targeted removal of aluminum through the in-situ application of lithium-fluoride salt (LiF), adopting a top-down strategy. In the initial step, the etching agent dissolves 3.2 grams of LiF in 40 mL of 9 M HCl. Following this, 2 g of the MAX powder was gently introduced to the etching agent and agitated for 24 hours at room temperature. Next, the MAX powder containing the etching agent was purified numerous times with DI water. This was accomplished by performing numerous centrifugation steps (each lasting 6 minutes at a speed of 3500 rpm) and sonication, with the slurry discarded post-centrifugation in every wash cycle. This procedure concluded once the solution attained a pH higher than 6 and observed a dark-green colloidal suspension of singular or few-layered (Ti3C2Tx) flakes. Obtained MXene colloidal solution was then preserved in a freezer at −20° C. to maintain its stability for future application. A free-standing MXene film was produced using vacuum-assisted filtration to process the Ti3C2Tx-rich colloidal solution through a filtration membrane (specifically, Celgard™ 3501 from Celgard LLC, USA). After being air-dried for 24 hours, the MXene (Ti3C2Tx) film was carefully detached from the filtration membrane. The concentration was found for MXene colloidal solution by dividing the weight of the air-dried MXene film by the volume of the colloidal solution. Finally, the dilution was performed to obtain a concentration of (1 mg/mL) of MXene colloidal solution using autoclaved double distilled water (18 MΩ).

### Synthesis of the Graphene Oxide

TRG is made by using Graphene oxide as a raw material and then thermally reducing it into a TRG powder. Graphene Oxide (GO) was synthesized using the Tour method: 300 mL of con·H2SO4 and 75 mL of orthophosphoric acid were mixed in a 500 ml beaker. Subsequently, 10.0 gm of graphite powder was added to the above solution and stir the mixture for 45 min at 30° C. Then KMnO4 was slowly (58.0g) added to the mixture. The mixture was allowed to stir at 25-30° C. for 72 h. Then 150 ml of 5% H2O2 solution was added to the mixture and stirred for 2 h at 30° C. The content was allowed to settle and centrifuge to remove the superannuated solution. The residue was washed with 1M HCl followed by DI water. The residue was further purified using cellulose membrane for 1 week in DI water medium. The purified residue was dried under the freeze drier, graphite oxide (GO) was obtained as a solid powder.

### Synthesis of Thermally Reduced Graphene (TRG) Solution

Thermally reduced graphene (TRG) was prepared by placing GO (300 mg) into a 25-mm i.d., 1.3-m long quartz tube. The opening end of the quartz tube was closed using a rubber stopper. A nitrogen inlet was then inserted through the rubber stopper. The sample was flushed with nitrogen for 15 min, and the quartz tube was quickly inserted into tube furnace, preheated to 1000° C. and held in the furnace for 30 s.

### Conducting Hydrogel Samples

Golde hydrogel solution in sol-state was placed on a hot-plate stirrer at 55° C. and stirred at 500 rpm. At a ratio of 10 v/v %, 0.5 mL of the conducting material solution was added (dropwise) into 5 mL of the Golde solution. The resulting solution was stirred for 10 minutes. At the same temperature, the stirring speed was lowered to 90 rpm to move bubbles to the surface (if any) for 10 minutes.

### All Flexible Electrode Fabrication

A 3D mold of the flexible electrode was designed using TinkerCAD™ and 3D printed into a flexible polyurethane mold using ELEEGOT Mars 3 pro 3D printer. The conducting hydrogel electrode was attached to a copper tape having a metal stud to connect to the ADInstruments Power lab™ 26T bio-amplifier, with only the head (active site) of the flexible electrode exposed to the skin, while the tail is covered to achieve a comparable result to the previously tested electrodes. Furthermore, in future tests and real-life applications, the setup would be furtherly improved and the exposed hydrogel area would be maximized to include the entire hydrogel area, thereby improving the signal quality. ADInstruments LabChart application was used to record and analyze the Lead I ECG signal.

### Characterization

### Morphological

To observe the hydrogel surface morphology, SEM imaging was conducted. To assess the available functional groups found throughout the hydrogel, Raman spectroscopy was used instead of FTIR since it is less sensitive to perturbations caused by the water molecules present in the hydrogel samples. Finally, since the material organization (i.e., amorphous, semicrystalline) in this hydrogel composite electrode affects surface morphology and, thereby its interactions with the skin (ex., wettability and contact area) in addition to its mechanical properties, XRD analysis was performed to improve our understanding of this material structure.

Sample preparation for analysis: In its sol-state the hydrogel samples were cast into a 3D printed mold of 6 mm diameter and 1.3 mm of thickness (refer to Figure S3). Next, the hydrogel is placed in a −20° C. freezer for 2 hours. Then the frozen samples are then rapidly moved into a freeze drier and placed there for 24 hours (until the sample is completely freeze-dried). Finally, the samples surfaces are gold coated to make them visible in the SEM scan. To perform the SEM imaging (JEOL JSM-7610F) SEM was used.

### Mechanical Properties

Hydrogel sample preparation: As distilled water is the main solvent of the hydrogels, all hydrogel samples were cast into a 3 cm×2 cm mold and soft-baked at 58° C. for around 10 minutes (an estimate based on observation) this is done to reduce the amount of the free water from the hydrogel matrix and in turn stabilizing it. This abovementioned temperature is not mandatory, however we observed that at lower temperatures the hydrogels was taking long time to be ready. At higher temperatures the hydrogels lost water rapidly (less controlled) and because it is believed that it could cause some of the conducting materials to start degrading. Then, they were left to cool down on a tabletop for 5 minutes. The tensile testing of the hydrogels as disclosed herein was performed in ambient room conditions to simulate the conditions that they will be exposed to during their application.

Tensile testing Using the Instron™ 5966 universal testing machine with a 10 kN load cell and an extension rate of 0.30 mm/s and 5N of maximum load, the hydrogels are fixed with a clamp sample holder. A small piece of tissue paper and duct tape were used to firmly attach the sample to the clamp without destroying the samples. The dog-bone-shaped samples were stretched to their breaking point while their stress/strain response was plotted.

### Electrical Properties

Electrochemical impedance spectroscopy (EIS) was carried out using a electrochemical impedance analyzer (AutoLab PGSTAT204, Sweden) with a sine wave with an amplitude of 10 mV and a frequency ranging from 0.1 Hz to 100 kHz with a focus window of 1 Hz to 100 kHz. The hydrogel sample was 16×14 mm and a thickness of 1.5 mm.

Cyclic voltammetry (CV) was performed on the same hydrogel samples at a scan rate of 0.05 and 0.1 V/s and a potential window of (±0.6 V). Specific capacitance was calculated from the CV data.

Gain response setup The gain response vs frequency of both the commercial and the hydrogel electrodes was evaluated using National Instrument tools, namely the NI ELVIS II+ board. The experimental protocol employed a sinusoidal signal of peak amplitude 0.1V and a frequency sweep starting from 1 Hz to-100 kHz as the stimulus.

Swelling ratio the hydrogels were placed in a pettri dish in ambient room conditions with an adequate amount of deionized water to submerge the hydrogels. Afterwards, the change in their weights was recorded using a balance and finally, the swelling ratio was estimated, this was performed in triplets (n=3)/variant over the course of 0.5, 1, 3, 5, 9, 12, 24 and 48 hours.

Adhesive test chicken skin preparation: chicken thighs were de-skinned, the skin was washed with 70% ethanol and dried off its oil and placed on a piece of aluminum foil.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

### REFERENCES CITED IN THE SPECIFICATION

(1) Kim, D.-H et al., J. A. Epidermal Electronics Science (1979) 2011, 333 (6044), 838-843.
(2) Yuk, H. et al., Hydrogel Bioelectronics. Chem Soc Rev 2019, 48 (6), 1642-1667.
(3) Liu, Y. et al.; Breathable, Self-Adhesive Dry Electrodes for Stable Electrophysiological Signal Monitoring During Exercise. ACS Appl Mater Interfaces 2022, 14 (10), 12812-12823.
(4) Li, L. et al.; Novel Highly Durable Carbon/Silver/Silver Chloride Composite Electrode for High-Definition Transcranial Direct Current Stimulation. Nanomaterials 2021, 11 (8), 1962.
(5) Carvalho, F. M. et al.; Nondrying, Sticky Hydrogels for the Next Generation of High-Resolution Conformable Bioelectronics. ACS Appl Electron Mater 2020, 2 (10), 3390-3401.
(6) Proctor, C. M. et al.; Ionic Hydrogel for Accelerated Dopamine Delivery via Retrodialysis; Chemistry of Materials 2019, 31 (17), 7080-7084.
(7) Liang, Q. et al.; Highly Stretchable Hydrogels as Wearable and Implantable Sensors for Recording Physiological and Brain Neural Signals. Advanced Science 2022, 9 (16).
(8) Roubert Martinez, S. et al.; Pure Conducting Polymer Hydrogels Increase Signal-to-Noise of Cutaneous Electrodes by Lowering Skin Interface Impedance. Adv Healthc Mater 2023, 12 (17).
(9) Correia, D. M. et al.; Ionic Liquid-Based Materials for Biomedical Applications. Nanomaterials 2021, 11 (9), 2401.
(10) Clasky, A. J. et al.; From Prevention to Diagnosis and Treatment: Biomedical Applications of Metal Nanoparticle-Hydrogel Composites; Acta Biomater 2021, 122, 1-25.
(11) Hardman, D. et al.; Self-Healing Ionic Gelatin/Glycerol Hydrogels for Strain Sensing Applications; NPG Asia Mater 2022, 14 (1), 11.
(12) Saleh, A. et al.; Inkjet-Printed Ti3C2Tx MXene Electrodes for Multimodal Cutaneous Biosensing. Journal of Physics: Materials 2020, 3 (4), 044004.
(13) Kaith, B. S. et al.; Hydrogels: Synthesis, Classification, Properties and Potential Applications-A Brief Review. J Polym Environ 2021, 29 (12), 3827-3841.
(14) Yang, G. et al.; Adhesive and Hydrophobic Bilayer Hydrogel Enabled On-Skin Biosensors for High-Fidelity Classification of Human Emotion. Adv Funct Mater 2022, 32 (29).
(15) Yuan, T. et al.; Highly Tough, Stretchable, Self-Healing, and Recyclable Hydrogels Reinforced by in Situ-Formed Polyelectrolyte Complex Nanoparticles. Macromolecules 2019, 52 (8), 3141-3149.
(16) Rebers, L. et al.; Differentiation of Physical and Chemical Cross-Linking in Gelatin Methacryloyl Hydrogels. Sci Rep 2021, 11 (1), 3256;
(17) Yang, X. et al.; Sandwich-like Polypyrrole/Reduced Graphene Oxide Nanosheets Integrated Gelatin Hydrogel as Mechanically and Thermally Sensitive Skinlike Bioelectronics; ACS Sustain Chem Eng 2020, acssuschemeng.0c01998.
(18) Wang, L. et al.; Biocompatible MXene/Chitosan-Based Flexible Bimodal Devices for Real-Time Pulse and Respiratory Rate Monitoring. ACS Mater Lett 2021, 3 (7), 921-929.
(19) Alizadeh, R. et al.; Conductive Hydrogels Based on Agarose/Alginate/Chitosan for Neural Disorder Therapy. Carbohydr Polym 2019, 224, 115161.
(20) Kang, B. et al.; Dual-Sensing, Stretchable, Fatigue-Resistant, Adhesive, and Conductive Hydrogels Used as Flexible Sensors for Human Motion Monitoring. Langmuir 2022, 38 (22), 7013-7023.
(21) Lee, Y. et al; Self-Adherent Biodegradable Gelatin-Based Hydrogel Electrodes for Electrocardiogramonitoring. Sensors 2020, 20 (20), 5737.
(22) Barleany, D. R. et al.; Synthesis and Characterization of Chitosan/Polyvinyl Alcohol Crosslinked Poly(N-Isopropylacrylamide) Smart Hydrogels via y-Radiation. Mater Today Proc 2023.
(23) Wang, Y. et al.; PVA/CMC/PEDOT:PSS Mixture Hydrogels with High Response and Low Impedance Electronic Signals for ECG Monitoring. Colloids Surf B Biointerfaces 2021, 208, 112088.
(24) Keller, A. et al.; Self-Healing Hydrogel Electrodes from Ingestible Materials. MRS Commun 2021, 11 (3), 342-348.
(25) Ying, B. et al.; Skin-like Hydrogel Devices for Wearable Sensing, Soft Robotics and Beyond. iScience 2021, 24 (11), 103174.
(26) Picos-Corrales, L. A.et al.; Chitosan as an Outstanding Polysaccharide Improving Health-Commodities of Humans and Environmental Protection. Polymers (Basel) 2023, 15 (3), 526.
(27) Zargar, V. et al.; A Review on Chitin and Chitosan Polymers: Structure, Chemistry, Solubility, Derivatives, and Applications. ChemBioEng Reviews 2015, 2 (3), 204-226.
(28) Li, Q. et al.; Ultrastretchable High-Conductivity MXene-Based Organohydrogels for Human Health Monitoring and Machine-Learning-Assisted Recognition. ACS Appl Mater Interfaces 2023, 15 (15), 19435-19446.
(29) Yin, H. et al.; Polyzwitterions as a Versatile Building Block of Tough Hydrogels: From Polyelectrolyte Complex Gels to Double-Network Gels. ACS Appl Mater Interfaces 2020, 12 (44), 50068-50076.
(30) Pan, Q. et al.; Biomolecule-Friendly Conducting PEDOT Interface for Long-Term Bioelectronic Devices. Sens Actuators B Chem 2022, 373, 132703.
(31) Nady, N. et al.; Novel Blend for Producing Porous Chitosan-Based Films Suitable for Biomedical Applications. Membranes (Basel) 2018, 8 (1), 2
(32) Wang, X. et al.; Mechanically Robust, Degradable and Conductive MXene-Composited Gelatin Organohydrogel with Environmental Stability and Self-Adhesiveness for Multifunctional Sensor. Compos B Eng 2022, 241, 110052.
(33) Riazi, H. et al.; Ti3C2 MXene-Polymer Nanocomposites and Their Applications. J Mater Chem A Mater 2021, 9 (13), 8051-8098.
(34) Alexander, C. K. et al.; Fundamentals of Electric Circuits; McGraw Hill Education, 2021; p 387.
(35) Hoon Lee, J. et al.; CNT/PDMS-Based Canal-Typed Ear Electrodes for Inconspicuous EEG Recording. J Neural Eng 2014, 11 (4), 046014.
(36) Akbar, T. F.et al.; Conductive Hydrogels for Bioelectronic Interfaces. In Neural Interface Engineering; Springer International Publishing: Cham, 2020; pp 237-265.
(37) Serrano, R. R. et al.; High Density Body Surface Potential Mapping with Conducting Polymer-Eutectogel Electrode Arrays for ECG Imaging. Advanced Science 2023.
(38) Arya, S. S. et al.; The Convergence of Traditional and Digital Biomarkers through AI-Assisted Biosensing: A New Era in Translational Diagnostics? Biosens Bioelectron 2023, 235, 115387.

What is claimed is:

1. A thermoreversible electrically and/or ionically conducting hydrogel comprising:
a mixture of glycerol, chitosan and gelatin,
a given amount of an electrically conducting material, and
an aqueous solvent;
wherein the conducting hydrogel is free of crosslinking agent.

2. The hydrogel of claim 1, wherein for 12 mL of said aqueous solvent, such as water, the hydrogel comprises:
about 12.5 to 30 v/v % of glycerol;
about 12.5 to 50 v/v % of a chitosan solution, wherein said chitosan solution comprises about 0.5 to 5 wt/Vol. % of chitosan in water with preferably 1 to 3 v/v of 99% acetic acid; and
about 14.5 to 58 wt.vol % of gelatin;
and about 10 v/v % of said conducting material.

3. The hydrogel of claim 1, wherein for 12 mL of said aqueous solvent, such as water, the hydrogel comprises:
about 1.5 to 3 mL of glycerol;
about 1.5 to 3 mL of a chitosan solution, wherein said chitosan solution comprises about 0.5 to 5 wt/Vol. % of chitosan in water with preferably 1 to 3 v/v of 99% acetic acid; and
about 1.75 to 3.75 g of gelatin;
and about 10 v/v % of said conducting material.

4. The hydrogel of claim 3, wherein for 12 mL of said aqueous solvent, such as water, the hydrogel comprises:
about 3 mL of glycerol;
about 3 mL of a chitosan solution, wherein said chitosan solution comprises about 0.5 to 5 wt/Vol. % of chitosan in water with preferably 1 to 3 v/v of 99% acetic acid; and
about 3.75 g of gelatin;
and about 10 v/v % of said conducting material.

5. The hydrogel of claim 1, wherein said conducting material comprises poly(3,4-ethylenedioxythiophene) polystyrene sulfonate (PEDOT:PSS), thermally reduced graphene (TRG), or Ti3C2Tx (MXene).

6. The hydrogel of claim 1, for use for the manufacturing of a medical electrode, a standalone transmitter for various signals, and/or wearable electronics for recording/stimulation.

7. The hydrogel of claim 6, wherein the medical electrode or standalone transmitter is manufactured using 3D printing, casting or molding techniques.

8. The hydrogel of claim 6, wherein the medical electrode is used for electrophysiological signal comprising electrocardiogramalectromyography (EMG), electroencephalography (EEG), electrogastrography (EGG) or electrooculography (EOG).

9. A process for the making of the electrically and/or ionically conducting hydrogel as claimed in claim 1, the process being a “one-pot” process and comprising the steps of:
heating an aqueous solvent, such as water, at a temperature between about 25 and 60° C.;
adding glycerol and chitosan into the heated solvent and stirring until to form a first mixture;
adding gelatin to the first mixture and stirring until to form a second mixture;
adding a given amount of an electrically conducting material, preferably in the form of salts or ionic liquid, to the second mixture and stirring until to form a third mixture; and
cooling the second or third mixture until the hydrogel is formed.

10. The process of claim 9, wherein the temperature of the aqueous solvent is about 50-60°, more preferably about 56° C.

11. The process of claim 9, wherein the aqueous solvent has a pH between about 4 and 6.5.

12. A medical electrode comprising:
the electrically and/or ionically conducting hydrogel as claimed in claim 1, the conducting hydrogel being in its gel state and having a shape configured for being applied to a patient's skin; and
an electrical connector, preferably comprising a metal or an intermediate flexible substrate, operatively connected to the electrically and/or ionically conducting hydrogel.

13. The medical electrode of claim 12, wherein the shape of the hydrogel is obtained by molding said hydrogel in its sol state at a given temperature, the hydrogel being then cooled until jellification/stretchable (Gel state).

14. The medical electrode of claim 12, wherein the shape of the hydrogel is obtained by 3D printing said hydrogel in its sol state at a given temperature.

15. The medical electrode of claim 12 for use for electrophysiological signal comprising electrocardiography (ECG), electromyography (EMG), electroencephalography (EEG), electrogastrography (EGG) or electrooculography (EOG).
